(19) United States
(12) Patent Application Publication
(10) Pub. No.: US 2017/0056336 A1
(43) Pub. Date: Mar. 2, 2017

(54) CO-TARGETING ANDROGEN RECEPTOR SPLICE VARIANTS AND MTOR SIGNALING PATHWAY FOR THE TREATMENT OF CASTRATION-RESISTANT PROSTATE CANCER

(71) Applicants: British Columbia Cancer Agency Branch, Vancouver (CA); British Columbia Cancer Agency Branch, Vancouver (CA)

(72) Inventors: Marianne Dorothy SADAR, West Vancouver (CA); Jun WANG, Surrey (CA); Nasrin R. MAWJI, Burnaby (CA); Minoru KATO, Ikoma (JP)

(21) Appl. No.: 15/149,298

(22) Filed: May 9, 2016

Related U.S. Application Data
(60) Provisional application No. 62/213,506, filed on Sep. 2, 2015.
(60) Provisional application No. 62/292,569, filed on Feb. 8, 2016.

(51) Int. Cl. A61K 31/09; A61K 31/4166; A61K 31/436; A61K 31/4745

(52) U.S. Cl. CPC A61K 31/09; A61K 31/4745; A61K 31/4166; A61K 31/436

(57) ABSTRACT

The present invention provides methods, compositions, and combinations for treating cancer via combined use of a compound of formula (I), and/or their subgenra, or a pharmaceutically acceptable salt, tautomer or stereoisomer thereof, wherein R¹, R², R³, R⁸, R⁹, R^{11a}, R^{11b}, R^{11c}, and R^{11d} are as defined herein, and at least one therapeutically active agents selected from inhibitors of PI3K/AKT/mTOR pathway, active agents associated with the treatment of prostate cancer, and anticancer agents.

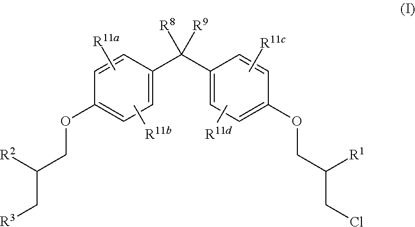

### CROSS-REFERENCE TO RELATED APPLICATION

This application claims the benefit of priority to U.S. Provisional Patent Application No. 62/213,506 filed Sep. 2, 2015 and U.S. Provisional Patent Application No. 62/292,569 filed Feb. 8, 2016, which are hereby incorporated by reference in their entirety for all purposes.

### STATEMENT OF GOVERNMENT INTEREST

This invention was made in part with government support under Grant No. 2R01 CA105304 awarded by the U.S. National Cancer Institute and Grant No. W81XWH-11-1-0551 awarded by the U.S. Department of Defense. The United States Government has certain rights in this invention.

### TECHNICAL FIELD

This invention generally relates to bisphenol-related compounds and their use for treatment of various indications in combination with another active agent. In particular the invention relates to bisphenol ether compounds and their use in combination with kinase inhibitors for treatment of various cancers, for example all stages of prostate cancer, including androgen dependent, androgen sensitive and castration-resistant prostate cancers. This invention also relates to bisphenol-related compounds and their use in combination with PI3K/mTOR dual inhibitor for treatment of various cancers.

### DESCRIPTION OF THE RELATED ART

Androgens mediate their effects through the androgen receptor (AR). Androgens play a role in a wide range of developmental and physiological responses and are involved in male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Several lines of evidence show that androgens are associated with the development of prostate carcinogenesis. Firstly, androgens induce prostatic carcinogenesis in rodent models (R. L. Noble, Cancer Res 37, 1929-1933 (1977); R. L. Noble, Oncology 34, 138-141 (1977)) and men receiving androgens in the form of anabolic steroids have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Secondly, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium while eliciting no effect on other male external genitalia (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration, known as androgen ablation therapy (ABT) or androgen depravation therapy (ADT).

Androgens also play a role in female diseases such as polycystic ovary syndrome as well as cancers. One example is ovarian cancer where elevated levels of androgens are associated with an increased risk of developing ovarian cancer (K. J. Helzlsouer, A. J. Alberg, G. B. Gordon, C. Longcope, T. L. Bush, S. C. Hoffman & G. W. Comstock, JAMA 274, 1926-1930 (1995); R. J. Edmondson, J. M. Monaghan & B. R. Davies, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit Rev Oncol Hematol 25, 1-9 (1997)), whereas estrogen receptor-alpha (ERa) and the progesterone receptor are detected in less than 50% of ovarian tumors.

The AR has distinct functional domains that include the carboxy-terminal ligand-binding domain (LBD), a DNA-binding domain (DBD) comprising two zinc finger motifs, and an N-terminus domain (NTD) that contains one or more transcriptional activation domains. Binding of androgen (ligand) to the LBD of the AR results in its activation such that the receptor can effectively bind to its specific DNA consensus site, termed the androgen response element (ARE), on the promoter and enhancer regions of “normally” androgen regulated genes, such as PSA, to initiate transcription. The AR can be activated in the absence of androgen by stimulation of the cAMP-dependent protein kinase (PKA) pathway, with interleukin-6 (IL-6) and by various growth factors (Culig et al 1994 Cancer Res. 54, 5474-5478; Nazareth et al 1996 J. Biol. Chem. 271, 19900-19907; Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The mechanism of ligand-independent transformation of the AR has been shown to involve: 1) increased nuclear AR protein suggesting nuclear translocation; 2) increased AR/ARE complex formation; and 3) the AR-NTD (Sadar 1999 J. Biol. Chem. 274, 7777-7783; Ueda et al 2002 A J. Biol. Chem. 277, 7076-7085; and Ueda et al 2002 B J. Biol. Chem. 277, 38087-38094). The AR can be activated in the absence of testicular androgens by alternative signal transduction pathways in castration-resistant disease, which is consistent with the finding that nuclear AR protein is present in secondary prostate cancer tumors (Kim et al 2002 Am. J. Pathol. 160, 219-226; and van der Kwast et al 1991 Inter. J. Cancer 48, 189-193).

Available inhibitors of the AR include nonsteroidal antiandrogens such as bicalutamide (Casodex™), nilutamide, flutamide, enzulutamide and investigational drug ARN-509 and steroidal antiandrogens, such as cyproterone acetate. These antiandrogens target the LBD of the AR and predominantly fail presumably due to poor affinity and mutations that lead to activation of the AR by these same antiandrogens (Taplin, M. E., Bubley, G. J., Kom Y. J., Small E. J., Uptonm M., Rajeshkumarm B., Balkm S. P., Cancer Res., 59, 2511-2515 (1999)). These antiandrogens would also have no effect on the recently discovered AR splice variants that lack the ligand-binding domain (LBD) to result in a constitutively active receptor which promotes progression of castration recurrent prostate cancer (CRPC) (Dehm S M, Schmidt L J, Heemers H V, Vessella R L, Tindall D J., Cancer Res 68, 5469-77, 2008; Guo Z, Yang X, Sun F, Jiang R, Linn D E, Chen H, Chen H, Kong X, Melamed J, Tepper C G, Kung H J, Brodie A M, Edwards J, Qiu Y., Cancer Res. 69, 2305-13, 2009; Hu et al 2009 Cancer Res. 69, 16-22; Sun et al 2010 J Clin Invest. 2010 120, 2715-30).

Conventional therapy has concentrated on androgen-dependent activation of the AR through its C-terminal domain. Studies developing antagonists to the AR have concentrated on the C-terminus and specifically: 1) the allosteric pocket and AF-2 activity (Estébanez-Perpiñá et al 2007, PNAS 104, 16074-16079); 2) in silico “drug repurposing” procedure for identification of nonsteroidal antagonists (Bisson et al 2007, PNAS 104, 11927-11932); and coactivator or corepressor interactions (Chang et al 2005, Mol Endocrinology 19, 2478-2490; Hur et al 2004, PLoS Biol 2, E274; Estébanez-Perpiñá et al 2005, JBC 280, 8060-8068; He et al 2004, Mol Cell 16, 425-438).

The AR-NTD is also a target for drug development (e.g. WO 2000/001813; Myung et al. J. Clin. Invest 2013, 123, 2948), since the NTD contains Activation-Function-1 (AF-1) which is the essential region required for AR transcriptional activity (Jenster et al 1991. Mol Endocrinol. 5, 1396-404). The AR-NTD importantly plays a role in activation of the AR in the absence of androgens (Sadar, M. D. 1999 J. Biol. Chem. 274, 7777-7783; Sadar M D et al 1999 Endocr Relat Cancer. 6, 487-502; Ueda et al 2002 J. Biol. Chem. 277, 7076-7085; Ueda 2002 J. Biol. Chem. 277, 38087-38094; Blaszczyk et al 2004 Clin Cancer Res. 10, 1860-9; Dehm et al 2006 J Biol Chem. 28, 27882-93; Gregory et al 2004 J Biol Chem. 279, 7119-30). The AR-NTD is important in hormonal progression of prostate cancer as shown by application of decoy molecules (Quayle et al 2007, Proc Natl Acad Sci USA. 104,1331-1336).

While the crystal structure has been resolved for the AR C-terminus LBD, this has not been the case for the NTD due to its high flexibility and intrinsic disorder in solution (Reid et al 2002 J. Biol. Chem. 277, 20079-20086) thereby hampering virtual docking drug discovery approaches. Compounds that modulate AR include the bis-phenol compounds disclosed in published PCT Nos: WO 2010/000066, WO 2011/082487; WO 2011/082488; WO 2012/145330; WO 2012/139039; WO 2012/145328; WO 2013/028572; WO 2013/028791; WO 2014/179867; and WO 2015/031984, which are hereby incorporated by reference in their entireties, to the British Columbia Cancer Agency Branch and The University of British Columbia.

The only effective treatment available for advanced prostate cancer is the withdrawal of androgens which are essential for the survival of prostate epithelial cells. ABT causes a temporary reduction in tumor burden concomitant with a decrease in serum prostate-specific antigen (PSA). Unfortunately prostate cancer can eventually grow again in the absence of testicular androgens (castration-resistant disease) (Huber et al 1987 Scand J. Urol Nephrol. 104, 33-39). Castration-resistant prostate cancer (CRPC) is biochemically characterized before the onset of symptoms by a rising titre of serum PSA (Miller et al 1992 J Urol. 147, 956-961). Once the disease becomes castration-resistant most patients succumb to their disease within two years.

Transcriptionally active androgen receptor plays a major role in CRPC in spite of reduced blood levels of androgen (Karantanos, T. et al Oncogene 2013, 32, 5501-5511; Harris, W. P. et al Nature Clinical Practice Urology, 2009, 6, 76-85). AR mechanisms of resistance to ADT include: overexpression of AR (Visakorpi, T. et al Nature Genetics 1995, 9, 401-406; Koivisto, P. et al Scandinavian Journal of Clinical and Laboratory Investigation Supplementum 1996, 226, 57-63); gain-of-function mutations in the AR LBD (Culig Z. et al Molecular Endocrinology 1993, 7, 1541-1550); intratumoral androgen synthesis (Cai, C. et al Cancer Research 2011, 71, 6503-6513); altered expression and function of AR coactivators (Ueda, T. et al The Journal of Biological Chemistry 2002, 277, 38087-38094; Xu J. et al Nature Reviews Cancer 2009, 9, 615-630); aberrant post-translational modifications of AR (Gioeli D. et al Molecular and Cellular Endocrinology 2012, 352, 70-78; van der Steen T. et al International Journal of Molecular Sciences 2013, 14, 14833-14859); and expression of AR splice variants (AR-Vs) which lack the ligand-binding domain (LBD) (Karantanos, T. et al Oncogene 2013, 32, 5501-5511; Andersen R. J. et al Cancer Cell 2010, 17, 535-546; Myung J. K. et al The Journal of Clinical Investigation 2013, 123, 2948-2960; Sun S. et al The Journal of Clinical Investigation 2010, 120, 2715-2730). Anti-androgens such as bicalutamide and enzalutamide target AR LBD, but have no effect on truncated constitutively active AR-Vs such as AR-V7 (Li Y. et al Cancer Research 2013, 73, 483-489). Expression of AR-V7 is associated with resistance to current hormone therapies (Li Y. et al Cancer Research 2013, 73, 483-489; Antonarakis E. S. et al The New England Journal of Medicine 2014, 371, 1028-1038).

Likewise, the PI3K/Akt/mTOR pathway is known to be a key oncogenic pathway in various cancers (Fruman D. A. et al Nature Reviews Drug Discovery 2014, 13, 140-156), and is linked to resistance to ADT in prostate cancers (Kaarbo M. et al Cellular Oncology: The Official Journal of the International Society for Cellular Oncology 2010, 32, 11-27). Alterations of components in PI3K/Akt/mTOR pathway occur in 42% of primary prostate tumors and 100% of metastatic tumors (Taylor B. S. et al Cancer Cell 2010, 18, 11-22). The PI3K/Akt/mTOR pathway is constitutively active due to loss of PTEN in the majority of advanced prostate cancers (McMenamin M. E. et al Cancer Research 1999, 59, 4291-4296). Targeting PI3K/Akt/mTOR is therefore considered a promising approach to treat CRPC (Bittig R. L. et al Endocrine-Related Cancer 2013, 20, R83-R99; Sarker D. et al Clinical Cancer Research. An Official Journal of the American Association of Cancer Research 2009, 15, 4799). However, the effects of inhibiting PI3K/Akt/mTOR signaling on AR are controversial (Carver B. S. et al Cancer Cell 2011, 19, 575-586; Mulholland D. J. et al Cancer Cell 2011, 19, 792-804; Liu, L. et al PloS One 2014, 9, e108780; Zhang W. et al Cancer Research 2009, 69, 7466-7472). There are numerous inhibitors that target PI3K/Akt/mTOR signaling such as rapamycin and its analogs, dual TORC1/2 inhibitors, pan-PI3K inhibitors, isoform-specific PI3K inhibitors, Akt inhibitors, and dual PI3K/TORC1/2 inhibitors.

While significant advances have been made in this field, there remains a need for improved therapy for treatment of cancer, especially castration-resistant prostate cancer. In particular, methods and compounds suitable for an effective cancer therapy, including combination therapy, for castration-resistant prostate cancer are needed. The present invention fulfills these needs, and provides other related advantages.

### BRIEF SUMMARY

In one embodiment, the present disclosure provides a pharmaceutical combination comprising a therapeutically effective amount of a compound of formula (I), or a pharmaceutically acceptable salt, tautomer or stereoisomer thereof; and at least one additional therapeutically active agent selected from the group consisting of inhibitors of PI3K/AKT/mTOR pathway, agents associated with the treatment of prostate cancer, and anticancer agents; wherein:

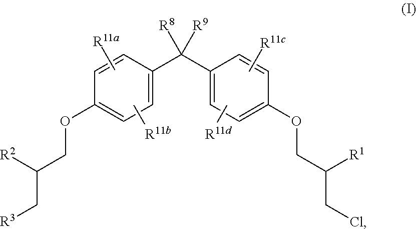

R¹ is hydroxyl or —OC(═O)R¹³;

R² is hydroxyl or —OC(═O)R¹³;

R³ is hydroxyl, halogen, or —OC(═O)R¹³;

R⁸ and R⁹ are each independently H, or C₁-C₃ alkyl;

R^{11a}, R^{11b}, R^{11c} and R^{11d} are each independently H or halogen;

R¹³ is C₁-C₆ alkyl; and

wherein, halo is selected from the group consisting of F, Cl, Br, and I.

In some embodiments, the pharmaceutical combination as described herein comprises one or more compounds selected from the group consisting of:

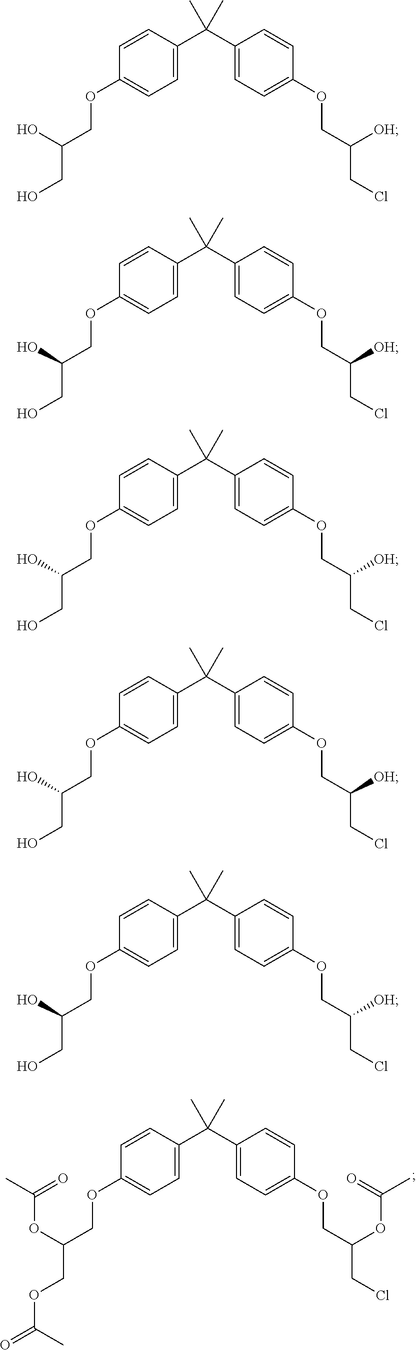 [SMILES: CC(C)(c1ccc(OCC(O)CO)cc1)c1ccc(OCC(O)CCl)cc1 ; CC(C)(c1ccc(OC[C@H](O)CO)cc1)c1ccc(OC[C@@H](O)CCl)cc1 ; CC(C)(c1ccc(OC[C@H](O)CCl)cc1)c1ccc(OC[C@@H](O)CO)cc1 ; CC(C)(c1ccc(OC[C@@H](O)CO)cc1)c1ccc(OC[C@@H](O)CCl)cc1 ; CC(C)(c1ccc(OC[C@H](O)CO)cc1)c1ccc(OC[C@H](O)CCl)cc1 ; CC(=O)OCC(COc1ccc(C(C)(C)c2ccc(OCC(CCl)OC(C)=O)cc2)cc1)OC(C)=O]

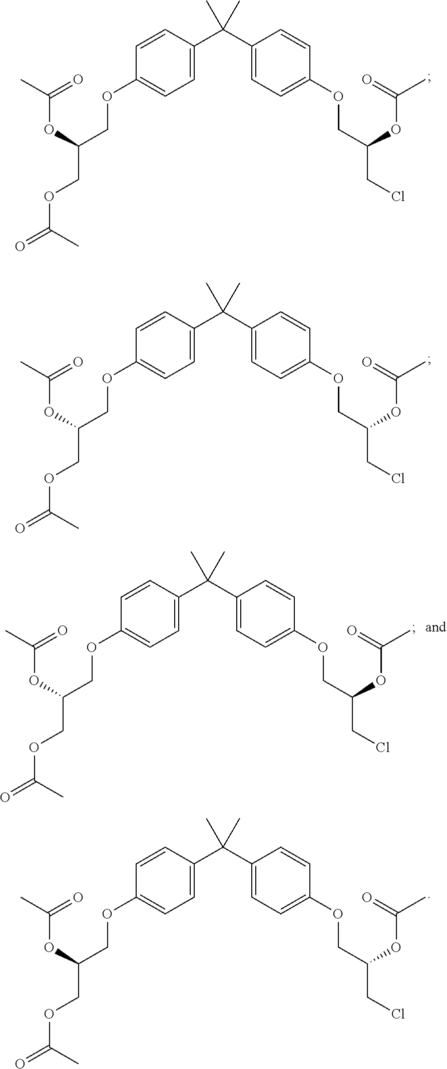

In one embodiment, a pharmaceutical combination is provided comprising a compound of formula (I) and/or their subgenra, or a pharmaceutically acceptable salt, tautomer or stereoisomer thereof, and the at least one additional therapeutically active agent in a single dosage form or in separate dosage forms. In some embodiments, the pharmaceutical combinations in separate dosage forms are administered via same mode of administration or different modes of administration. In other embodiments, the pharmaceutical combination in separate dosage forms are co-administered via simultaneous administration, sequential administration, overlapping administration, interval administration, continuous administration, or a combination thereof.

In one embodiment, a pharmaceutical combination is provided comprising a compound of formula (I) and/or their subgenra, or a pharmaceutically acceptable salt, tautomer or stereoisomer thereof, and at least one additional therapeutically active agent selected from inhibitors of PI3K/AKT/mTOR pathway. In one embodiment, said inhibitor of PI3K/AKT/mTOR pathway is a dual PI3K/mTOR inhibitor. In some embodiments, the dual PI3K/mTOR inhibitor is selected from the group consisting of: BEZ-235 (Dactolisib), BEZ-235, XL-765, PF-4691502, GSK-2126458, GDC-0980 and PKI-587. In one embodiment, the dual PI3K/mTOR inhibitor is BEZ-235.

In one embodiment, a pharmaceutical combination is provided comprising a compound of formula (I) and/or their subgenra, or a pharmaceutically acceptable salt, tautomer or stereoisomer thereof, and at least one additional therapeutically active agent in a pharmaceutical formulation further comprising a pharmaceutically acceptable excipient or a pharmaceutically acceptable carrier.

In other embodiments, a pharmaceutical combination is provided comprising a compound of formula (I) and/or their subgenra, or a pharmaceutically acceptable salt, tautomer or stereoisomer thereof, and at least one additional therapeutically active agent, further comprising a second additional therapeutically active agent. In one embodiment, said second additional therapeutically active agent is selected from the group consisting of: selected from the group consisting of enzalutamide, galeterone, ARN-509 (4-(7-(6-cyano-5-(tlifluoromethyl)pylidin-3-yl)-8-oxo-6-thioxo-5,7-diazaspiro[3,4]octan-5-yl)-2-tluoro-N-methylbenzamide), abiraterone, bicalutamide, nilutamide, flutamide, cyproterone acetate, docetaxel, bevacizumab, OSU-HDAC42 ((S)-(+)-N-Hydroxy-4-(3-methy-2-phenyl-butyrylamino)benzamide, monoclonal antibody against the vascular integrin ανβ3, sunitumib, ZD-4054 (zibotentan), cabazitaxel (XRP-6258), MDX-010 (ipilimumab), OGX 427 (apatorsen), OGX 011 (custirsen), finasteride, dutasteride, turosteride, bexlosteride, izonsteride, FCE 28260 ((1S,3aS,3bS,5aR,9aR,9bS,11aS)-9a,11a-dimethyl-7-oxo-N-(1,1,1-trifluoro-2-phenylpropan-2-yl)-1,2,3,3a,3b,4,5,5a,6,9b,10,11-dodecahydroindeno[5,4-f]quinoline-1-carboxamide), SKF105,111 (1β-(Di-isopropyl-aminocarbonyl)androsta-3,5-diene-3-carboxylic acid), radium 233, ODM-201, and related compounds thereof.

In one embodiment, the present disclosure provides a method for treating a condition or disease that is responsive to modulation of androgen receptor activity, comprising administering to the subject, a therapeutically effective amount of a compound of formula (I);

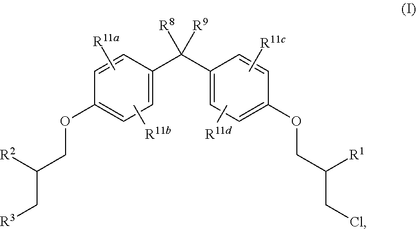

or a pharmaceutically acceptable salt, tautomer or stereoisomer thereof;
wherein

R¹ is hydroxyl or —OC(═O)R¹³;

R² is hydroxyl or —OC(═O)R¹³;

R³ is hydroxyl, halogen, or —OC(═O)R¹³;

R⁸ and R⁹ are each independently H, or C₁-C₃ alkyl;

R^{11a}, R^{11b}, R^{11c} and R^{11d} are each independently H or halogen;

R¹³ is C₁-C₆ alkyl; and

wherein, halo is selected from the group consisting of F, Cl, Br, and I;

and administering of at least one additional therapeutically active agent selected from P inhibitors of PI3K/AKT/mTOR pathway, agents associated with the treatment of prostate cancer, and anticancer agents, before, during, or after the subject has been administered a compound of formula (I).

In one embodiment, the method for treating a condition or disease that is responsive to modulation of androgen receptor activity comprising administering to the subject, a therapeutically effective amount of a compound of formula (I) and/or their subgenra, or a pharmaceutically acceptable salt, tautomer or stereoisomer thereof, and at least one additional therapeutically active agent, is for treating conditions or diseases is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration.

In some embodiments, the method for treating a condition or disease that is responsive to modulation of androgen receptor activity comprising administering to the subject, a therapeutically effective amount of a compound of formula (I) and/or their subgenra, or a pharmaceutically acceptable salt, tautomer or stereoisomer thereof, and at least one additional therapeutically active agent, is for treating prostate cancer. In one embodiment, said prostate cancer is castration resistant prostate cancer. In some embodiment, said prostate cancer is androgen-dependent prostate cancer or androgen-independent prostate cancer. In some embodiments, the method for treating a condition of disease that is responsive to modulation of androgen receptor activity as described herein is for treating breast cancer.

In one embodiment of the present disclosure, a method is provided for reducing or preventing tumor growth, comprising contacting tumor cells with a therapeutically effective amount of a compound of formula (I);

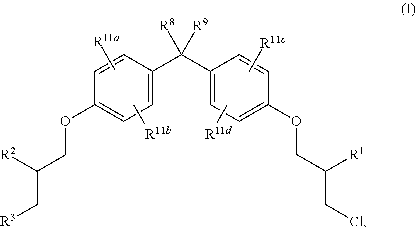

or a pharmaceutically acceptable salt, tautomer or stereoisomer thereof;
wherein

R¹ is hydroxyl or —OC(═O)R¹³;

R² is hydroxyl or —OC(═O)R¹³;

R³ is hydroxyl, halogen, or —OC(═O)R¹³;

R⁸ and R⁹ are each independently H, or C₁-C₃ alkyl;

R^{11a}, R^{11b}, R^{11c} and R^{11d} are each independently H or halogen;

R¹³ is C₁-C₆ alkyl; and

wherein, halo is selected from the group consisting of F, Cl, Br, and I;

and contacting of at least one additional therapeutically active agent selected from inhibitors of PI3K/AKT/mTOR pathway, agents associated with the treatment of prostate cancer, and anticancer agents, before, during, or after the subject has been administered a compound of formula (I).

In one embodiment, the method for reducing or preventing tumor growth as described herein, is to treat tumor cell is selected from the group consisting of: prostate cancer, breast cancer, ovarian cancer, endometrial cancer, and salivary gland carcinoma. In some embodiments, the method for reducing or preventing tumor growth as described herein, is for treating tumor of the prostate cancer. In one embodiment, said prostate cancer is castration resistant prostate cancer. In some embodiment, said prostate cancer is androgen-dependent prostate cancer or androgen-independent prostate cancer. In some embodiments, the educing or preventing tumor growth as described herein is for treating tumor of the breast cancer.

In one embodiment, the method for reducing or preventing tumor growth as described herein is in vivo or in vitro.

### DESCRIPTION OF THE FIGURES

FIG. 1A shows comparative expression levels of p110 isoforms, pAkt, and pS6 in cell lines.

FIG. 1B shows the analyses of the pAkt levels in LNCaP95 cells where the expression levels of p110 β (siB1,2,3) or p110 δ (siD1,2,3) were knocked down for 48 h.

FIG. 1C shows titration experiments of BEZ-235 (BEZ) and everolimus.

FIG. 1D shows the effect of enzalutamide (ENZ), Compound A (EPI) and BEZ-235 on mTOR and AR pathways in LNCaP95 cells.

FIG. 1E shows how the effect of enzalutamide (ENZ), Compound A (EPI) and BEZ-235 on mTOR and AR pathways in parental LNCaP cells.

FIG. 2A shows the effect of LNCaP95 cells transiently transfected with PSA-, ARR3- or PB-luciferase reporters were treated with DMSO, Compound A (EPI), enzalutamide (ENZ), BEZ-235 (BEZ) or combination for 1 hr prior to the addition of R1881 for 48 hr in serum-free conditions. LNCaP95 cells transfected with PSA-luciferase reporter were also treated with everolimus (10 nM) or combination with enzalutamide or Compound A to compare with results using BEZ-235.

FIG. 2B shows the effect of LNCaP cells transiently transfected with PSA-, ARR3- or PB-luciferase reporters were treated with DMSO, Compound A, enzalutamide, BEZ-235 or combination for 1 hr prior to the addition of R1881 for 48 hr in serum-free conditions.

FIG. 2C shows the luciferase activity of the Cos-1 cells which were co-transfected with PB-luciferase, and expression vectors for ARv567 or ARV7 for 5 h, and then treated with DMSO, Compound A, BEZ-235 or combination of Compound A and BEZ-235 for 24 h in serum-free conditions.

FIG. 2D shows transactivation assays of the AR-NTD in LNCaP and LNCaP95 cells cotransfected with p5xGal4UAS-TATA-luciferase and AR NTD-Gal4DBD. Compound A, BEZ-235, or combination of Compound A and BEZ-235 were added 1 h before addition of IL-6 (50 ng/ml) or FSK (50 uM) in LNCaP cells and harvested after 24 h. LNCaP95 cells were harvested 24 h after the treatment of indicated compound.

FIG. 3A shows transcript levels of FL-AR regulated genes KLK3, FKBP5, and TMPRSS2 in a LNCaP95 cell assay.

FIG. 3B shows transcript levels of AR-V7 regulated genes UBE2C, CDC20, and Akt1 in a LNCaP95 cell assay.

FIG. 3C shows transcript levels of FL-AR and AR-V7 in a LNCaP95 cell assay.

FIG. 4A shows measurement of LNCaP95 cell proliferation which were treated with DMSO, Compound A (EPI), enzalutamide (ENZ), BEZ-235 (BEZ) or combination of Compound A and BEZ-235 for 1 h prior to the addition of R1881 (0.1 nM) for 48 h in serum-free media.

FIG. 4B shows measurement of LNCaP95 cell proliferation which were treated with everolimus (EVE, 10 nM) instead with BEZ-235.

FIG. 4C shows the degree of tumor growth in castrated mice which were administered a vehicle, a half-dose of Compound A (100 mg/kg body weight), BEZ-235 (5 mg/kg body weight) or combination thereof, daily by oral gavage for two weeks.

FIG. 4D shows the body weight change over the duration of the experiment shown in FIG. 4C.

FIG. 4E shows Western blot analyses of protein lysates from xenografts harvested 1 h after the last treatment as demonstrated in FIG. 4C. Three xenografts from each treatment group are shown. P3-Actin was used as a loading control. The ratios of phosphoprotein to total protein are shown for pAkt ser473, pS6 and p4EBP1.

FIG. 5A shows immunohistochemistry of representative xenografts stained for hematoxylin and eosin (H-E), UBE2C, pS6, Ki-67 and TUNEL.

FIG. 5B shows % Ki67 positive cell counts in sections from xenografts for each treatment.

FIG. 5C shows % TUNEL positive cell counts in sections from xenografts for each treatment.

FIG. 6A depicts a hypothetical model where Compound A (EPI) inhibits transcriptional activity of FL-AR and AR-Vs which results in reduced expression of target genes such as PSA and UBE2C respectively. Compound A reduces mTOR-regulated pS6 by inhibiting AR-regulation of mTOR.

FIG. 6B depicts a hypothetical model where mTOR inhibitors such as everolimus (EVE) and low dose of BEZ-235 blocking mTOR with concomitant increases of FL-AR and AR-V. Increased levels of FL-AR lead to increased levels of expression of its target gene, PSA.

FIG. 6C depicts a hypothetical model where increased transcriptional activity of FL-AR due to increased levels in response to mTOR inhibition is blocked by AR-NTD antagonist. Combination of AR-NTD antagonist (Compound A) and mTOR inhibitor blocks mTOR-regulated pS6 and transcriptional activity of AR-V7 to reduce levels of its target gene, UBE2C. driven UBE2C.

### DETAILED DESCRIPTION

### I. Definitions

In the following description, certain specific details are set forth in order to provide a thorough understanding of various embodiments. However, one skilled in the art will understand that the invention can be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH₂ radical.

“Cyano” refers to the —CN radical.

“Halo” or “halogen” refers to bromo, chloro, fluoro or iodo radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C1-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” refers to a radical of the formula —R_{b}—R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl. cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heterocyclylalkyl” refers to a radical of the formula —R_{b}—Rₑ where R_{b} is an alkylene, alkenylene, or alkynylene chain as defined above and Rₑ is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene, alkenylene, or alkynylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)N R_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

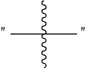

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

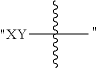

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

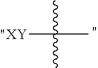

infers that when R³ is “XY”, the point of attachment bond is the same bond as the bond by which R³ is depicted as being bonded to CH₃.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring can be replaced with a nitrogen atom.

The invention disclosed herein is also meant to encompass the in vivo metabolic products of the disclosed compounds. Such products can result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

As used herein, a “subject” can be a human, non-human primate, mammal, rat, mouse, cow, horse, pig, sheep, goat, dog, cat and the like. The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art. The terms “subject” and “patient” are used interchangeably throughout the present application.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances can or can not occur, and that the description includes instances where said event or circumstance occurs and instances in which it does not. For example, “optionally substituted aryl” means that the aryl radical can or can not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

Compounds as described herein can be in the free form or in the form of a salt thereof. In some embodiments, compounds as described herein can be in the form of a pharmaceutically acceptable salt, which are known in the art (Berge et al., J. Pharm. Sci. 1977, 66, 1). “Pharmaceutically acceptable salt” as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt can be, for example, formed as a pharmaceutically acceptable salt. Compounds containing one or more basic functional groups can be capable of forming a “pharmaceutically acceptable acid addition salt” with, for example, a pharmaceutically acceptable organic or inorganic acid. Pharmaceutically acceptable salts can be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid.

Compounds containing one or more acidic functional groups can be capable of forming “Pharmaceutically acceptable base addition salt” with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts can be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, 1-ephenamine, N,N-dibenzylethylenediamine or polyamine resins. In some embodiments, compounds as described herein can contain both acidic and basic groups and can be in the form of inner salts or zwitterions, for example, and without limitation, betaines. Salts as described herein can be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts can occur in situ during isolation and purification of the compounds or preparation of salts can occur by separately reacting an isolated and purified compound.

Often crystallizations produce a solvate of the compound of the invention. As used herein, the term “solvate” refers to an aggregate that comprises one or more molecules of a compound of the invention with one or more molecules of solvent. The solvent can be water, in which case the solvate can be a hydrate. Alternatively, the solvent can be an organic solvent. Thus, the compounds of the present invention can exist as a hydrate, including a monohydrate, dihydrate, hemihydrate, sesquihydrate, trihydrate, tetrahydrate and the like, as well as the corresponding solvated forms. The compound of the invention can be true solvates, while in other cases, the compound of the invention can merely retain adventitious water or be a mixture of water plus some adventitious solvent.

In some embodiments, compounds and all different forms thereof (e.g. free forms, salts, polymorphs, isomeric forms) as described herein can be in the solvent addition form, for example, solvates. Solvates contain either stoichiometric or non-stoichiometric amounts of a solvent in physical association the compound or salt thereof. The solvent can be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein can include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature can cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formula illustrated for the sake of convenience.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount can be less than a therapeutically effective amount.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” can be used interchangeably or can be different in that the particular malady or condition can not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 software naming program (CambridgeSoft). For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

Throughout the present specification, the terms “about” and/or “approximately” can be used in conjunction with numerical values and/or ranges. The term “about” is understood to mean those values near to a recited value. For example, “about 40 [units]” can mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein. The terms “about” and “approximately” can be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

### II. Compounds

The present invention provides bisphenol-related compounds of formula (I), and/or their subgenra, or a pharmaceutically acceptable salt, tautomer or stereoisomer thereof. The compounds disclosed herein can be used in a combination with at least one additional therapeutically active agent (combination therapy). The bisphenol-related compounds as disclosed herein were developed to specifically target the AR amino-terminal domain (NTD) to block the transcriptional activities of FL-AR (full-length androgen receptor) and AR-Vs (AR-splice variants), which results in antitumor activity in CRPC xenografts (Andersen, R. J. et. al., Cancer Cell 2010, 17, 535-546; Myung, J. K. et al., J. Clin. Invest. 2013, 123, 2948-2960; Martin, S. K. et al. Molecular Oncology 2015, 9, 628-639).

As noted above, certain embodiments of the present invention are directed to compounds useful for treatment of various cancers, including various types of prostate cancers. While not wishing to be bound by any theory, it is believed that binding of the compounds to the androgen receptor (for example at the N-terminal domain) can contribute to the activity of the disclosed compounds.

In one embodiment the invention includes compounds which form covalent bonds with the androgen receptor (AR) (e.g., at the N-terminal domain), thus resulting in irreversible (or substantially irreversible) inhibition of the same. In this regard, the certain compounds of the present invention are designed to include functional groups capable of forming covalent bonds with a nucleophile under certain in vivo conditions. For example, in some embodiments the reactivity of compounds of the present invention is such that they will not substantially react with various nucleophiles (e.g., glutathione) when the compounds are free in solution. However, when the free mobility of the compounds is restricted, and an appropriate nucleophile is brought into close proximity to the compound, for example when the compounds associate with, or bind to, the androgen receptor, the compounds are capable of forming covalent bonds with certain nucleophiles (e.g., thiols).

The present invention includes all compounds which have the above described properties (i.e., binding to androgen receptor (AR)). In one embodiment, the present invention is directed to a compound having a structure of Formula (I):

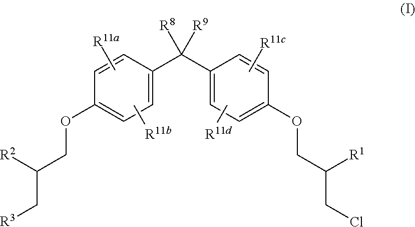

or a pharmaceutically acceptable salt, tautomer or stereoisomer thereof, wherein:

R¹ is hydroxyl or —OC(═O)R¹³;

R² is hydroxyl or —OC(═O)R¹³;

R³ is hydroxyl, halogen, or —OC(═O)R¹³;

R⁸ and R⁹ are each independently H, or C₁-C₃ alkyl;

R^{11a}, R^{11b}, R^{11c} and R^{11d} are each independently H or halogen;

R¹³ is C₁-C₆ alkyl; and

wherein, halo is selected from the group consisting of F, Cl, Br, and I.

In one embodiment, R¹ is hydroxyl or —OC(═O)CH₃.

In one embodiment, R² is hydroxyl or —OC(═O)CH₃.

In one embodiment, R³ is hydroxyl or —OC(═O)CH₃.

In one embodiment, R¹, R², and R³ are each hydroxyl. In another embodiment, R¹, R², and R³ are each —OC(═O)CH₃.

In one embodiment, R⁸ and R⁹ are each methyl.

In one embodiment, R^{11a}, R^{11b}, R^{11c} and R^{11d} are each H. In another embodiment, one of R^{11a}, R^{11b}, R^{11c} and R^{11d} is halogen and the remaining three substituents are each H. In some embodiments, two of R^{11a}, R^{11b}, R^{11c} and R^{11d} are each halogen, and the remaining two substituents are each H.

In one embodiment, R¹³ is methyl. In one embodiment, R¹³ is ethyl.

In some more specific embodiments of the compound of Formula (I) is a racemate.

In another embodiment, the compound of Formula (I) is a stereoisomer where the stereochemistry at the carbon atoms bearing R¹ and R² are defined as (S) or (R).

In some specific embodiments of the compound of Formula I, the compound has one of the following structures:

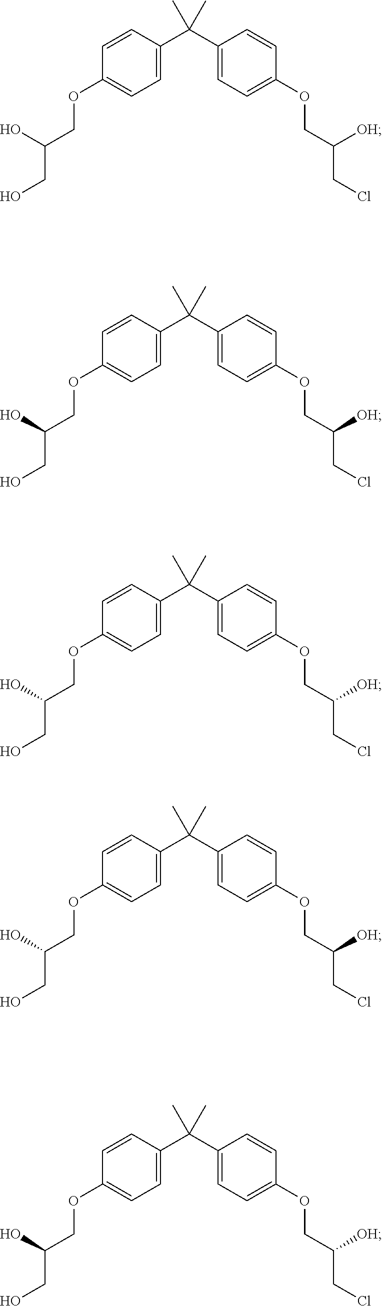 [SMILES: CC(C)(c1ccc(OCC(O)CO)cc1)c1ccc(OCC(O)CCl)cc1 ; CC(C)(c1ccc(OC[C@H](O)CO)cc1)c1ccc(OC[C@@H](O)CCl)cc1 ; CC(C)(c1ccc(OC[C@H](O)CCl)cc1)c1ccc(OC[C@@H](O)CO)cc1 ; CC(C)(c1ccc(OC[C@@H](O)CO)cc1)c1ccc(OC[C@@H](O)CCl)cc1 ; CC(C)(c1ccc(OC[C@H](O)CO)cc1)c1ccc(OC[C@H](O)CCl)cc1]

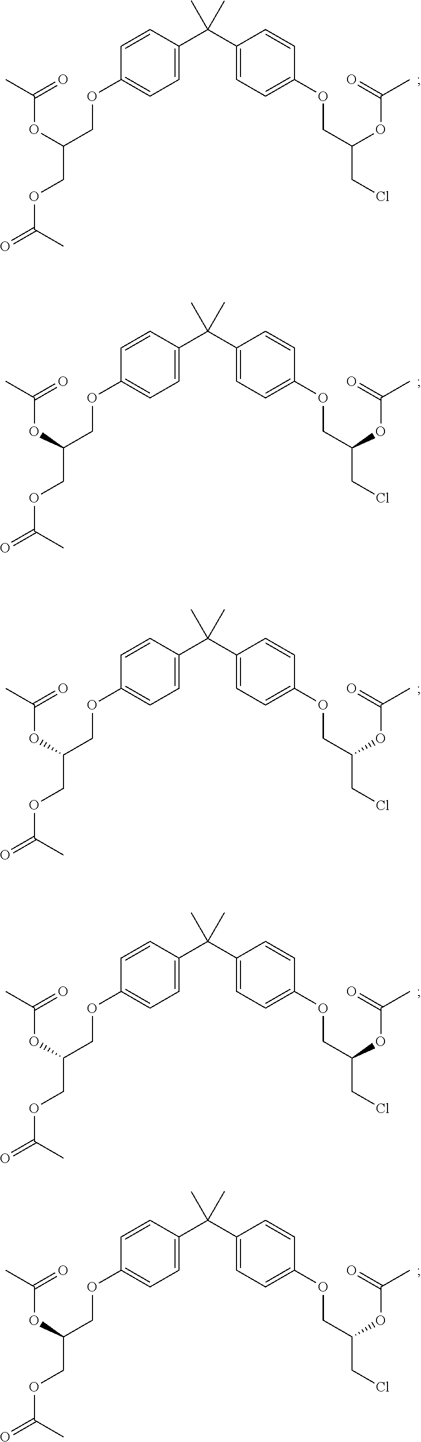 [SMILES: CC(=O)OCC(COc1ccc(C(C)(C)c2ccc(OCC(CCl)OC(C)=O)cc2)cc1)OC(C)=O ; CC(=O)OC[C@H](COc1ccc(C(C)(C)c2ccc(OC[C@H](CCl)OC(C)=O)cc2)cc1)OC(C)=O ; CC(=O)OC[C@@H](COc1ccc(C(C)(C)c2ccc(OC[C@@H](CCl)OC(C)=O)cc2)cc1)OC(C)=O ; CC(=O)OC[C@@H](COc1ccc(C(C)(C)c2ccc(OC[C@H](CCl)OC(C)=O)cc2)cc1)OC(C)=O ; CC(=O)OC[C@H](COc1ccc(C(C)(C)c2ccc(OC[C@@H](CCl)OC(C)=O)cc2)cc1)OC(C)=O]

or a pharmaceutically acceptable salt, tautomer, or stereoisomer thereof.

### III. Additional Therapeutically Active Agents

In one embodiment, the present invention provides a combination therapy comprising a compound of formula (I), and/or their subgenra, or a pharmaceutically acceptable salt, tautomer or stereoisomer thereof. The following therapeutically active agents may be employed in conjunction with the administration of the compounds described above.

### Inhibitors of the PI3K/AKT/mTOR Pathway

PI3K/AKT/mTOR pathway is an intracellular signaling pathway important to the life cycle of cells. Thus, PI3K/AKT/mTOR pathway plays an important role in cell proliferation which is implicated in various diseases including cancer, e.g., prostate cancer. Phosphatidylinositol-3-kinase (P13K) activation leads to phosphorylation of the protein kinase B (AKT) which signals cell growth. One of the downstream effects of the activation of PI3K/AKT is the activation of the mammalian target of rapamycin (mTOR). mTOR is responsible for regulation of cellular metabolism, growth and proliferation. Hence, over-activation of the PI3K/AKT/mTOR pathway could result in unregulated cell proliferation such as cancer cell growth or tumor growth. PI3K/AKT/mTOR pathway is implicated as a potential driver of castration resistant prostate cancer, in addition to androgen receptor (Bitting, R. L. et. al. Endocrine-Related Cancer 2013, 20, R83-99; Zhang, W. et al. Cancer Research 2009, 69, 7466-7472; Edlind M. P. et al. Asian Journal of Andrology 2014, 16, 378-386).

In one embodiment, a compound of formula (I), and/or their subgenra, or a pharmaceutically acceptable salt, tautomer or stereoisomer thereof, is administered in combination with an inhibitor of PI3K/AKT/mTOR pathway. In some embodiments, the combination therapy of a compound of formula (I), and/or their subgenra, or a pharmaceutically acceptable salt, tautomer or stereoisomer thereof, and an inhibitor of PI3K/AKT/mTOR pathway is beneficial in the treatment of disease or disorder associated with cell proliferation, such as cancer.

In one embodiment, PI3K/AKT/mTOR pathway inhibitors include, but are not limited to, BEZ-235 (2-methyl-2-[4-(3-methyl-2-oxo-8-quinolin-3-ylimidazo[4,5-c]quinolin-1-yl)phenyl]propanenitrile), SF-1126 ((3S)-4-[[(1S)-1-carboxy-2-hydroxyethyl]amino]-3-[[2-[[(2S)-5-(diaminomethylideneamino)-2-[[4-oxo-4-[[4-(4-oxo-8-phenylchromen-2-yl)morpholin-4-ium-4-yl]methoxy]butanoyl]amino]pentanoyl]amino]acetyl]amino]-4-oxobutanoate), XL147 (N-[3-(2,1,3-benzothiadiazol-5-ylamino)quinoxalin-2-yl]-4-methylbenzenesulfonamide), XL418 (1-[3-[4-(3-bromo-2H-pyrazolo[3,4-d]pyrimidin-4-yl)piperazin-1-yl]-4-methyl-5-(2-pyrrolidin-1-ylethylamino)phenyl]-4,4,4-trifluorobutan-1-one), XL-147 (Pilaralisib; 2-amino-N-[3-[[3-(2-chloro-5-methoxyanilino)quinoxalin-2-yl]sulfamoyl]phenyl]-2-methylpropanamide), GDC-0941 (4-[2-(1H-indazol-4-yl)-6-[(4-methylsulfonylpiperazin-1-yl)methyl]thieno[3,2-d]pyrimidin-4-yl]morpholine), LY294002 (2-Morpholin-4-yl-8-phenylchromen-4-one), LY29002 (celecoxib and analogs thereof, wortmannin, 17-hydroxywortmanninm PI-103 (3-(4-morpholin-4-ylpyrido[2,3]furo[2,4-b]pyrimidin-2-yl)phenol.), BKM120 (buparlisib; 5-(2,6-dimorpholin-4-ylpyrimidin-4-yl)-4-(trifluoromethyl)pyridin-2-amine or buparlisib HCl; 5-(2,6-dimorpholin-4-ylpyrimidin-4-yl)-4-(trifluoromethyl)pyridin-2-amine hydrochloride), CAL-101 (idelalisib; 5-fluoro-3-phenyl-2-[(1S)-1-(7H-purin-6-ylamino)propyl]quinazolin-4-one), CAL-263, IC-87114 (2-[(6-aminopurin-9-yl)methyl]-5-methyl-3-(2-methylphenyl)quinazolin-4-one), GSK2636771 (2-methyl-1-[[2-methyl-3-(trifluoromethyl)phenyl]methyl]-6-morpholin-4-ylbenzimidazole-4-carboxylic acid), TG 100713 (3-(2,4-diaminopteridin-6-yl)phenol), BYL719 (alpelisib; (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide), 3-methyladenine, YM201636 (6-amino-N-[3-(4-morpholin-4-ylpyrido[2,3]furo[2,4-b]pyrimidin-2-yl)phenyl]pyridine-3-carboxamide or 6-amino-N-[3-(4-morpholin-4-ylpyrido[2,3]furo[2,4-b]pyrimidin-2-yl)phenyl]pyridine-3-carboxamide hydrochloride), NVP-BGT226 ((Z)-but-2-enedioic acid; 8-(6-methoxypyridin-3-yl)-3-methyl-1-[4-piperazin-1-yl-3-(trifluoromethyl)phenyl]imidazo[4,5-c]quinolin-2-one), BAY80-6946 (copanlisib; 2-amino-N-[7-methoxy-8-(3-morpholin-4-ylpropoxy)-2,3-dihydroimidazo[1,2-c]quinazolin-5-yl]pyrimidine-5-carboxamide), PF-04691502 (2-amino-8-[4-(2-hydroxyethoxy)cyclohexyl]-6-(6-methoxypyridin-3-yl)-4-methylpyrido[2,3-d]pyrimidin-7-one), PKI-402 (1-[4-(3-ethyl-7-morpholin-4-yltriazolo[4,5-d]pyrimidin-5-yl)phenyl]-3-[4-(4-methylpiperazine-1-carbonyl)phenyl]urea), CH5132799 (5-(7-methylsulfonyl-2-morpholin-4-yl-5,6-dihydropyrrolo[2,3-d]pyrimidin-4-yl)pyrimidin-2-amine), GDC-0980 (1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one or Apitolisib; (2S)-1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one), NU7026 (2-morpholin-4-ylbenzo[h]chromen-4-one), NU7441 (8-dibenzothiophen-4-yl-2-morpholin-4-ylchromen-4-one), AS-252424 ((5Z)-5-[[5-(4-fluoro-2-hydroxyphenyl)furan-2-yl]methylidene]-1,3-thiazolidine-2,4-dione or (5E)-5-[[5-(4-fluoro-2-hydroxyphenyl)furan-2-yl]methylidene]-1,3-thiazolidine-2,4-dione), AS-604850 (5-[(2,2-difluoro-1,3-benzodioxol-5-yl)methylidene]-1,3-thiazolidine-2,4-dione, or (5E)-5-[(2,2-difluoro-1,3-benzodioxol-5-yl)methylidene]-1,3-thiazolidine-2,4-dione or (5Z)-5-[(2,2-difluoro-1,3-benzodioxol-5-yl)methylidene]-1,3-thiazolidine-2,4-dione), AS-041164 (5-(1,3-benzodioxol-5-ylmethylidene)-1,3-thiazolidine-2,4-dione), CAY10505 ((5E)-5-[[5-(4-fluorophenyl)furan-2-yl]methylidene]-1,3-thiazolidine-2,4-dione), GSK2126458 (Omipalisib; 2,4-difluoro-N-[2-methoxy-5-(4-pyridazin-4-ylquinolin-6-yl)pyridin-3-yl]benzenesulfonamide), A66 ((2S)-1-N-[5-(2-tert-butyl-1,3-thiazol-4-yl)-4-methyl-1,3-thiazol-2-yl]pyrrolidine-1,2-dicarboxamide), PF-05212384 (PKI-587; gedatolisib; 1-[4-[4-(dimethylamino)piperidine-1-carbonyl]phenyl]-3-[4-(4,6-dimorpholin-4-yl-1,3,5-triazin-2-yl)phenyl]urea), PIK-294 (2-[[4-amino-3-(3-hydroxyphenyl)pyrazolo[3,4-d]pyrimidin-1-yl]methyl]-5-methyl-3-(2-methylphenyl)quinazolin-4-one), PIK-293 (2-[(4-aminopyrazolo[3,4-d]pyrimidin-1-yl)methyl]-5-methyl-3-(2-methylphenyl)quinazolin-4-one), XL765 (N-[4-[[3-(3,5-dimethoxyanilino)quinoxalin-2-yl]sulfamoyl]phenyl]-3-methoxy-4-methylbenzamide), PIK-93 (N-[5-[4-chloro-3-(2-hydroxyethylsulfamoyl)phenyl]-4-methyl-1,3-thiazol-2-yl]acetamide), AZD6482 (2-[[(1R)-1-(7-methyl-2-morpholin-4-yl-4-oxopyrido[1,2-a]pyrimidin-9-yl)ethyl]amino]benzoic acid), AS-605240 ((5Z)-5-(quinoxalin-6-ylmethylidene)-1,3-thiazolidine-2,4-dione), GSK1059615 ((5Z)-5-[(4-pyridin-4-ylquinolin-6-yl)methylidene]-1,3-thiazolidine-2,4-dione), TG100-115 (3-[2,4-diamino-7-(3-hydroxyphenyl)pteridin-6-yl]phenol), PIK-75 (N-[(E)-(6-bromoimidazo[1,2-a]pyridin-3-yl)methylideneamino]-N,2-dimethyl-5-nitrobenzenesulfonamide hydrochloride or N-[(E)-(6-bromoimidazo[1,2-a]pyridin-3-yl)methylideneamino]-N,2-dimethyl-5-nitrobenzenesulfonamide), PIK-90 (N-(7,8-dimethoxy-2,3-dihydroimidazo[1,2-c]quinazolin-5-yl)pyridine-3-carboxamide), TGX-115 (8-(2-methylphenoxy)-2-morpholin-4-yl-1H-quinolin-4-one), TGX-221 (9-(1-anilinoethyl)-7-methyl-2-morpholin-4-ylpyrido[1,2-a]pyrimidin-4-one), ZSTK474 (4-[4-[2-(difluoromethyl)benzimidazol-1-yl]-6-morpholin-4-yl-1,3,5-triazin-2-yl]morpholine), MK-2206 (8-[4-(1-aminocyclobutyl)phenyl]-9-phenyl-2H-[1,2,4]triazolo[3,4-f][1,6]naphthyridin-3-one or 8-[4-(1-aminocyclobutyl)phenyl]-9-phenyl-2H-[1,2,4]triazolo[3,4-f][1,6]naphthyridin-3-one dihydrochloride), quercetin, tetrodotoxin citrate, thioperamide maieate, deguelin, (−)-deguelin, OSU03012 (2-amino-N-[4-[5-phenanthren-2-yl-3-(trifluoromethyl)pyrazol-1-yl]phenyl]acetamide), PI 828 (8-(4-aminophenyl)-2-morpholin-4-ylchromen-4-one), WHI-P154 (2-bromo-4-[(6,7-dimethoxyquinazolin-4-yl)amino]phenol), INK-1117 ([6-(2-amino-1,3-benzoxazol-5-yl)imidazo[1,2-a]pyridin-3-yl]-morpholin-4-ylmethanone), IPI-145 (duvelisib; 8-chloro-2-phenyl-3-[(1S)-1-(7H-purin-6-ylamino)ethyl]isoquinolin-1-one), PP121 (1-cyclopentyl-3-(1H-pyrrolo[2,3-b]pyridin-5-yl)pyrazolo[3,4-d]pyrimidin-4-amine), PX-478 (4-[(2S)-2-amino-2-carboxyethyl]-N,N-bis(2-chloroethyl)benzeneamine oxide dihydrochloride), PX-866, PX-867 ([(3aR,6E,9S,9aR,10R,11aS)-5-hydroxy-9-(methoxymethyl)-9a,11a-dimethyl-1,4,7-trioxo-6-(pyrrolidin-1-ylmethylidene)-2,3,3a,9,10,11-hexahydroindeno[4,5-h]isochromen-10-yl] acetate), P529 (palomid-529; 8-(1-hydroxyethyl)-2-methoxy-3-[(4-methoxyphenyl)methoxy]benzo[c]chromen-6-one), GNE-477 (5-[7-methyl-6-[(4-methylsulfonylpiperazin-1-yl)methyl]-4-morpholin-4-ylthieno[3,2-d]pyrimidin-2-yl]pyrimidin-2-amine), CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide), WAY-266176 ([(1S,3aR,6E,9S,9aR,10R,11aS)-6-[[3-(dimethylamino)propyl-methylamino]methylidene]-1,5-dihydroxy-9-(methoxymethyl)-9a,11a-dimethyl-4,7-dioxo-2,3,3a,9, 10,11-hexahydro-1H-indeno[4,5-h]isochromen-10-yl] acetate), WAY-266175, LME00084, IC486068, PWT33597 (VDC-597), triciribine, tandutinib (4-[6-methoxy-7-(3-piperidin-1-ylpropoxy)quinazolin-4-yl]-N-(4-propan-2-yloxyphenyl)piperazine-1-carboxamide), temsirolimus ((1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate), deforolimus (ridaforolimus; (1R,2R,4S)-4-[(2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate), everolimus (dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone], zotarolimus ((3S,6R,7E,9R,10R,12R,14S,15E,17E,19E,21S,23S,26R,27R,34aS)-9,27-dihydroxy-10,21-dimethoxy-3-{(1R)-2-[(1S,3R,4S)-3-methoxy-4-(1H-tetrazol-1-yl)cyclohexyl]-1-methylethyl}-6,8,12,14,20,26-hexamethyl-4,9,10,12,13,14,21,22,23,24,25,26,27,32,33,34,34a-heptadecahydro-3H-23,27-epoxypyrido[2,1-c][1,4]oxazacyclohentriacontine-1,5,11,28,29(6H,31H)-pentone), and umirolimus (Biolimus A9).

In some embodiments, the inhibitor of the PI3K/AKT/mTOR pathway is a PI3K/AKT/mTOR dual inhibitor. In one embodiment, the PI3K/mTOR dual inhibitors include, but are not limited to, BEZ-235, XL-765, PF-4691502, GSK-2126458, GDC-0980 and PKI-587. In another embodiment, the PI3K/mTOR dual inhibitor is BEZ-235.

### Inhibitors of Androgen Receptor of Ligand Binding Domain (LBD)

Androgen receptor (AR) is implicated as a potential driver of castration resistant prostate cancer. In one embodiment, a compound of formula (I), and/or their subgenra, or a pharmaceutically acceptable salt, tautomer or stereoisomer thereof, is administered in combination with an inhibitor of AR LBD. In some embodiments, the combination therapy of a compound of formula (I), and/or their subgenra, or a pharmaceutically acceptable salt, tautomer or stereoisomer thereof, and an inhibitor of AR LBD is beneficial in the treatment of disease or disorder associated with cell proliferation, such as cancer.

In one embodiment, inhibitors of AR LBD include, but are not limited to bicalutamide (Casodex™; (N-[4-cyano-3-(trifluoromethyl)phenyl]-3-[(4-fluorophenyl)sulfonyl]-2-hydroxy-2-methylpropanamide), nilutamide (5,5-dimethyl-3-[4-nitro-3-(trifluoromethyl)phenyl] imidazolidine-2,4-dione), flutamide (2-methyl-N-[4-nitro-3-(trifluoromethyl)phenyl]-propanamide), galeterone, enzalutamide (4-(3-(4-cyano-3-(trifluoromethyl)phenyl)-5, 5-dimethyl-4-oxo-2-thioxoimidazolidin-1-yl)-2-fluoro-N-methylbenzamide), apalutamide (ARN-509), ODM-201 (BAY-1841788), abiraterone (or CB-7630; (3S,8R,9S,10R,13S,14S)-10,13-dimethyl-17-(pyridin-3-yl) 2,3,4,7,8,9,10,11,12,13,14,15-dodecahydro-1H-cyclopenta[a]phenanthren-3-ol), and steroidal antiandrogens such as cyproterone acetate (6-chloro-1β,2β-dihydro-17-hydroxy-3′H-cyclopropa[1,2]pregna-4,6-diene-3,20-dione).

Agents Associated with the Treatment of Prostate Cancer

In one embodiment, a compound of formula (I), and/or their subgenra, or a pharmaceutically acceptable salt, tautomer or stereoisomer thereof, is administered in combination with an active agent associated with the treatment of prostate cancer. In some embodiments, the combination therapy of a compound of formula (I), and/or their subgenra, or a pharmaceutically acceptable salt, tautomer or stereoisomer thereof, and an active agent associated with the treatment of prostate cancer is beneficial in the treatment of disease or disorder associated with cell proliferation.

In one embodiment, active agents associated with the treatment of prostate cancer include, but are not limited to docetaxel (Taxotere; 1,70,100-trihydroxy-9-oxo-5,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}), Bevacizumab (Avastin), OSU-HDAC42 ((S)-(+)-N-hydroxy-4-(3-methyl-2-phenylbutyrylamino)-benzamide), VITAXIN, sunitumib (N-(2-diethylaminoethyl)-5-[(Z)-(5-fluoro-2-oxo-1H-indol-3-ylidene)methyl]-2,4-dimethyl-1H-pyrrole-3-carboxamide), ZD-4054 (N-(3-Methoxy-5-methylpyrazin-2-yl)-2-[4-(1,3,4-oxadiazol-2-yl)phenyl]pyridin-3-sulfonamid), Cabazitaxel (XRP-6258), MDX-010 (Ipilimumab), OGX 011, finasteride (Proscar, Propecia; N-(1,1-dimethylethyl)-3-oxo-(5a,17(3)-4-azaandrost-1-ene-17-carboxamide), dutasteride (Avodart; 5α,17β)-N-{2,5 bis(trifluoromethyl) phenyl}-3-oxo-4-azaandrost-1-ene-17-carboxamide), turosteride ((4aR,4bS,6aS,7S,9aS,9bS,11aR)-1,4a,6a-trimethyl-2-oxo-N-(propan-2-yl)-N-(propan-2 ylcarbamoyl)hexadecahydro-1H-indeno[5,4-f]quinoline-7-carboxamide), bexlosteride (LY-191,704; (4aS,10bR)-8-chloro-4-methyl-1,2,4a,5,6,10b-hexahydrobenzo[f]quinolin-3-one), izonsteride (LY-320,236; (4aR,10bR)-8-[(4-ethyl-1,3-benzothiazol-2-yl)sulfanyl]-4,10b-dimethyl-1,4,4a,5,6,10b-hexahydrobenzo[f]quinolin-3 (2H)-one), FCE 28260, and SKF105,111.

### Anticancer Agents

In addition to agents associated with the treatment of prostate cancer, other anticancer agents in some embodiments are used in combination with the compounds of the present application. Anticancer agents may include agents selected from any of the classes known to those of ordinary skill in the art, including, for example, alkylating agents, anti-metabolites, plant alkaloids and terpenoids (e.g., taxanes), topoisomerase inhibitors, anti-tumor antibiotics, kinase inhibitors, hormonal therapies, molecular targeted agents, and the like. Generally such an anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor an Hsp90 inhibitor, a CDK (cyclin-dependent kinase) inhibitor, CHK (checkpoint kinase) inhibitor, PARP (poly (DP-ribose)polymerase) inhibitors, and the like.

Alkylating agents include (a) alkylating-like platinum-based chemotherapeutic agents such as cisplatin, carboplatin, nedaplatin, oxaliplatin, satraplatin, and (SP-4-3)-(cis)-amminedichloro-[2-methylpyridine] platinum(II); (b) alkyl sulfonates such as busulfan; (c) ethyleneimine and methylmelamine derivatives such as altretamine and thiotepa; (d) nitrogen mustards such as chlorambucil, cyclophosphamide, estramustine, ifosfamide, mechlorethamine, trofosamide, prednimustine, melphalan, and uramustine; (e) nitrosoureas such as carmustine, lomustine, fotemustine, nimustine, ranimustine and streptozocin; (f) triazenes and imidazotetrazines such as dacarbazine, procarbazine, temozolamide, and temozolomide.

Anti-metabolites include (a) purine analogs such as fludarabine, cladribine, chlorodeoxyadenosine, clofarabine, mercaptopurine, pentostatin, and thioguanine; (b) pyrimidine analogs such as fluorouracil, gemcitabine, capecitabine, cytarabine, azacitidine, edatrexate, floxuridine, and troxacitabine; (c) antifolates, such as methotrexate, pemetrexed, raltitrexed, and trimetrexate. Anti-metabolites also include thymidylate synthase inhibitors, such as fluorouracil, raltitrexed, capecitabine, floxuridine and pemetrexed; and ribonucleotide reductase inhibitors such as claribine, clofarabine and fludarabine.

Plant alkaloid and terpenoid derived agents include mitotic inhibitors such as the vinca alkaloids vinblastine, vincristine, vindesine, and vinorelbine; and microtubule polymer stabilizers such as the taxanes, including, but not limited to paclitaxel, docetaxel, larotaxel, ortataxel, and tesetaxel.

Topoisomerase inhibitors include topoisomerase I inhibitors such as camptothecin, topotecan, irinotecan, rubitecan, and belotecan; and topoisomerase II inhibitors such as etoposide, teniposide, and amsacrine.

Anti-tumor antibiotics include (a) anthracyclines such as daunorubicin (including liposomal daunorubicin), doxorubicin (including liposomal doxorubicin), epirubicin, idarubicin, and valrubicin; (b) streptomyces-related agents such as bleomycin, actinomycin, mithramycin, mitomycin, porfiromycin; and (c) anthracenediones, such as mitoxantrone and pixantrone. Anthracyclines have three mechanisms of action: intercalating between base pairs of the DNA/RNA strand; inhibiting topoiosomerase II enzyme; and creating iron-mediated free oxygen radicals that damage the DNA and cell membranes. Anthracyclines are generally characterized as topoisomerase II inhibitors.

Hormonal therapies include (a) androgens such as fluoxymesterone and testolactone; (b) antiandrogens such as bicalutamide, cyproterone, flutamide, and nilutamide; (c) aromatase inhibitors such as aminoglutethimide, anastrozole, exemestane, formestane, and letrozole; (d) corticosteroids such as dexamethasone and prednisone; (e) estrogens such as diethylstilbestrol; (f) antiestrogens such as fulvestrant, raloxifene, tamoxifen, and toremifine; (g) LHRH agonists and antagonists such as buserelin, goserelin, leuprolide, and triptorelin; (h) progestins such as medroxyprogesterone acetate and megestrol acetate; and (i) thyroid hormones such as levothyroxine and liothyronine.

Molecular targeted agents include (a) receptor tyrosine kinase (‘RTK’) inhibitors, such as inhibitors of EGFR, including erlotinib, gefitinib, and neratinib; inhibitors of VEGFR including vandetanib, semaxinib, and cediranib; and inhibitors of PDGFR; further included are RTK inhibitors that act at multiple receptor sites such as lapatinib, which inhibits both EGFR and HER2, as well as those inhibitors that act at of each of C-kit, PDGFR and VEGFR, including but not limited to axitinib, sunitinib, sorafenib and toceranib; also included are inhibitors of BCR-ABL, c-kit and PDGFR, such as imatinib; (b) FKBP binding agents, such as an immunosuppressive macrolide antibiotic, including bafilomycin, rapamycin (sirolimus) and everolimus; (c) gene therapy agents, antisense therapy agents, and gene expression modulators such as the retinoids and rexinoids, e.g. adapalene, bexarotene, trans-retinoic acid, 9 cis retinoic acid, and N (4 hydroxyphenyl)retinamide; (d) phenotype-directed therapy agents, including: monoclonal antibodies such as alemtuzumab, bevacizumab, cetuximab, ibritumomab tiuxetan, rituximab, and trastuzumab; (e) immunotoxins such as gemtuzumab ozogamicin; (f) radioimmunoconjugates such as 131I-tositumomab; and (g) cancer vaccines.

HDAC inhibitors include, but are not limited to, (i) hydroxamic acids such as Trichostatin A, vorinostat (suberoylanilide hydroxamic acid (SAHA)), panobinostat (LBH589) and belinostat (PXD101) (ii) cyclic peptides, such as trapoxin B, and depsipeptides, such as romidepsin (NSC 630176), (iii) benzamides, such as MS-275 (3-pyridylmethyl-N-{4-[(2-aminophenyl)-carbamoyl]-benzyl}-carbamate), CI994 (4-acetylamino-N-(2aminophenyl)-benzamide) and MGCD0103 (N-(2-aminophenyl)-4-((4-(pyridin-3-yl)pyrimidin-2-ylamino)methyl)benzamide), (iv) electrophilic ketones, (v) the aliphatic acid compounds such as phenylbutyrate and valproic acid.

Hsp90 inhibitors include, but are not limited to, benzoquinone ansamycins such as geldanamycin, 17 DMAG (17-Dimethylamino-ethylamino-17-demethoxygeldanamycin), tanespimycin (17 AAG, 17-allylamino-17 demethoxygeldanamycin), EC5, retaspimycin (IPI-504, 18,21 didehydro-17-demethoxy-18,21-dideoxo-18,21-dihydroxy-17-(2 propenylamino)-geldanamycin), and herbimycin; pyrazoles such as CCT 018159 (4-[4-(2,3-dihydro-1,4-benzodioxin-6-yl)-5-methyl-1H-pyrazol-3-yl]-6-ethyl-1,3-benzenediol); macrolides, such as radicocol; as well as BIIB021 (CNF2024), SNX-5422, STA-9090, and AUY922.

CDK inhibitors include, but are not limited to, AZD-5438, BMI-1040, BMS-032, BMS-387, CVT-2584, flavopyridol, GPC-286199, MCS-5A, PD0332991, PHA-690509, seliciclib (CYC202, R-roscovitine), ZK-304709 AT7519M, P276-00, SCH 727965, AG-024322, LEE011, LY2835219, P1446A-05, BAY 1000394, SNS-032. and the like.

CHK inhibitors include, but are not limited to, 5-(3-fluorophenyl)-3-ureidothiophene-N—[(S)-piperidin-3-yl]-2-carboxamide (AZD7762), 7-nitro-1H-indole-2-carboxylic acid {4-[1-(guanidinohydrazone)-ethyl]-phenyl}-amide (PV1019), 5-[(8-chloro-3-isoquinolinyl)amino]-3-[(1R)-2-(dimethylamino)-1-methylethoxy]-2-pyrazinecarbonitrile (SAR-020106), PF-00477736, CCT241533, 6-bromo-3-(1-methyl-1H-pyrazol-4-yl)-5-(3R)-3-piperidinyl-pyrazolo[1,5-a]pyrimidin-7-amine (SCH900776), 7-hydroxystaurosporine (UCN-01), 4-[((3S)-1-azabicyclo[2.2.2]oct-3-yl)amino]-3-(1H-benzimidazol-2-yl)-6-chloroquinolin-2(1H)-one (CHIR 124), 7-aminodactinomycin (7-AAD), isogranulatimide, debromohymenialdisine, N-[5-Bromo-4-methyl-2-[(2S)-2-morpholinylmethoxy]-phenyl]-N′-(5-methyl-2-pyrazinyl)urea) (LY2603618), sulforaphane (4-methylsulfinylbutyl isothiocyanate), 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078), TAT-S216A (synthetic peptide; YGRKKRRQRRRLYRSPAMPENL), CBP501 ((d-Bpa)sws(d-Phe-F5)(d-Cha)rrrqrr), and the like.

PARP inhibitors include, but are not limited to, 4-(3-(1-(cyclopropanecarbonyl)piperazine-4-carbonyl)-4-fluorobenzyl)phthalazin-1 (2H)-one (olaparib, AZD2281, Ku-0059436), 2-[(2R)-2-methylpyrrolidin-2-yl]-1H-benzimidazole-4-carboxamide (Veliparib, ABT-888), (8S,9R)-5-fluoro-8-(4-fluorophenyl)-9-(1-methyl-1H-1,2,4-triazol-5-yl)-8,9-dihydro-2H-pyrido[4,3,2-de]phthalazin-3 (7H)-one (talazoparib, BMN 673), 4-iodo-3-nitrobenzamide (iniparib, BSI-201), 8-fluoro-5-(4-((methylamino)methyl)phenyl)-3,4-dihydro-2H-azepino[5,4,3-cd]indol-1(6H)-one phosphoric acid (Rucaparib, AG-014699, PF-01367338), 2-[4-[(dimethylamino)methyl]phenyl]-5,6-dihydroimidazo[4,5,1-jk][1,4]benzodiazepin-7(4H)-one (AG14361), 3-aminobenzamide (INO-1001), 2-(2-fluoro-4-((S)-pyrrolidin-2-yl)phenyl)-3H-benzo[d]imidazole-4-carboxamide (A-966492), N-(5,6-dihydro-6-oxo-2-phenanthridinyl)-2-acetamide hydrochloride (PJ34, PJ34 HCl), MK-4827, 3,4-dihydro-4-oxo-3,4-dihydro-4-oxo-N-[(1S)-1-phenylethyl]-2-quinazolinepropanamide (ME0328), 5-(2-oxo-2-phenylethoxy)-1(2H)-isoquinolinone (UPF-1069), 4-[[4-fluoro-3-[(4-methoxy-1-piperidinyl)carbonyl]phenyl]methyl]-1(2H)-phthalazinone (AZD 2461), and the like.

Miscellaneous agents include altretamine, arsenic trioxide, gallium nitrate, hydroxyurea, levamisole, mitotane, octreotide, procarbazine, suramin, thalidomide, photodynamic compounds such as methoxsalen and sodium porfimer, and proteasome inhibitors such as bortezomib.

Biologic therapy agents include: interferons such as interferon-α2a and interferon-α2b, and interleukins such as aldesleukin, denileukin diftitox, and oprelvekin.

In addition to anticancer agents intended to act against cancer cells, combination therapies including the use of protective or adjunctive agents, including: cytoprotective agents such as armifostine, dexrazonxane, and mesna, phosphonates such as parmidronate and zoledronic acid, and stimulating factors such as epoetin, darbeopetin, filgrastim, PEG-filgrastim, and sargramostim, are also envisioned.

### IV. Combination Therapy

One embodiment comprises the use of the disclosed compounds in combination therapy with one or more currently-used or experimental pharmacological therapies which are utilized for treating the above disease states irrespective of the biological mechanism of action of such pharmacological therapies, including without limitation pharmacological therapies which directly or indirectly inhibit the androgen receptor, pharmacological therapies which are cyto-toxic in nature, and pharmacological therapies which interfere with the biological production or function of androgen (hereinafter, an “additional therapeutic agent”). By “combination therapy” is meant the administration of any one or more of a compound of formula (I), and/or their subgenra, or a pharmaceutically acceptable salt, tautomer or stereoisomer thereof with one or more additional therapeutically active agent to the same patient such that their pharmacological effects are contemporaneous with one another, or if not contemporaneous, that their effects are synergistic with one another even though dosed sequentially rather than contemporaneously.

In one embodiment, the present invention provides a method of treating a condition associated with cell proliferation in a patient in need thereof. In one embodiment, the present invention provides a method of treating cancer or tumors. In another embodiment, the present invention provides a method of treating prostate cancer or breast cancer. The method comprises co-administering to a patient in need thereof a therapeutically effective amount of at least one compound of formula (I), and/or their subgenra, or a pharmaceutically acceptable salt, tautomer or stereoisomer thereof and at least one additional therapeutically active agent. In some embodiment, at least one additional therapeutically active agent is selected from the group consisting of inhibitors of PI3K/AKT/mTOR pathway, agents associated with the treatment of prostate cancer, and anticancer agents. The term “patient” or “subject” as used herein, includes humans and animals, preferably mammals.

In one embodiment, the compound of formula (I), and/or their subgenra, or a pharmaceutically acceptable salt, tautomer or stereoisomer thereof, is administered at a dose from about 5 mg/day to about 500 mg/day. In one embodiment, at least one additional therapeutically active agent is administered at about 1 mg/day to about 500 mg/day. In another embodiment, the compound of formula (I), and/or their subgenra, or a pharmaceutically acceptable salt, tautomer or stereoisomer thereof and/or at least one additional therapeutically active agent is administered at a dose from about 1 mg/m² to about 3 g/m², from about 5 mg/m² to about 1 g/m², or from about 10 mg/m² to about 500 mg/m².

The administered dose may be expressed in units of mg/m²/day in which a patient's body surface area (BSA) may be calculated in m² using various available formulae using the patient's height and weight. The administered dose may alternatively be expressed in units of mg/day which does not take into consideration the patient's BSA. It is straightforward to convert from one unit to another given a patient's height and weight.

The term “co-administration” or “coadministration” refers to administration of (a) a compound of formula (I), and/or their subgenra, or a pharmaceutically acceptable salt, tautomer or stereoisomer thereof and (b) at least one additional therapeutically active agent, together in a coordinated fashion. For example, the co-administration can be simultaneous administration, sequential administration, overlapping administration, interval administration, continuous administration, or a combination thereof. In one embodiment, a compound of formula (I), and/or their subgenra, or a pharmaceutically acceptable salt, tautomer or stereoisomer thereof and at least one additional therapeutically active agent are formulated into a single dosage form. In another embodiment, the compound of formula (I), and/or their subgenra, or a pharmaceutically acceptable salt, tautomer or stereoisomer thereof and at least one additional therapeutically active agent are provided in a separate dosage forms.

In one embodiment, the co-administration is carried out for one or more treatment cycles. By “treatment cycle”, it is meant a pre-determined period of time for co-administering the compound of formula (I), and/or their subgenra, or a pharmaceutically acceptable salt, tautomer or stereoisomer thereof and at least one therapeutically active agent. Typically, the patient is examined at the end of each treatment cycle to evaluate the effect of the present combination therapy. In one embodiment, the co-administration is carried out for 1 to 48 treatment cycles. In another embodiment, the co-administration is carried out for 1 to 36 treatment cycles. In another embodiment, the co-administration is carried out for 1 to 24 treatment cycles.

In one embodiment, each of the treatment cycle has about 3 or more days. In another embodiment, each of the treatment cycle has from about 3 days to about 60 days. In another embodiment, each of the treatment cycle has from about 5 days to about 50 days. In another embodiment, each of the treatment cycle has from about 7 days to about 28 days. In another embodiment, each of the treatment cycle has 28 days. In one embodiment, the treatment cycle has about 29 days. In another embodiment, the treatment cycle has about 30 days. In another embodiment, the treatment cycle has about a month-long treatment cycle. In another embodiment, the treatment cycle has from about 4 to about 6 weeks.

Depending on the patient's condition and the intended therapeutic effect, the dosing frequency for each of the c compound of formula (I), and/or their subgenra, or a pharmaceutically acceptable salt, tautomer or stereoisomer thereof and at least one therapeutically active agent may vary from once per day to six times per day. That is, the dosing frequency may be once per day, twice per day, three times per day, four times per day, five times per day, or six times per day. In some embodiments, dosing frequency may be one to six times per week or one to four times per month. In one embodiment, dosing frequency may be once a week, once every two weeks, once every three weeks, once every four weeks, or once a month.

There may be one or more void days in a treatment cycle. By “void day”, it is meant a day when neither the compound of formula (I), and/or their subgenra, or a pharmaceutically acceptable salt, tautomer or stereoisomer thereof or at least one therapeutically active agent is administered. In other words, none of the compound of formula (I), and/or their subgenra, or a pharmaceutically acceptable salt, tautomer or stereoisomer thereof and at least one therapeutically active agent is administered on a void day. Any treatment cycle must have at least one non-void day. By “non-void day”, it is meant a day when at least one of the compound of formula (I), and/or their subgenra, or a pharmaceutically acceptable salt, tautomer or stereoisomer thereof and at least one therapeutically active agent is administered.

By “simultaneous administration”, it is meant that the compound of formula (I), and/or their subgenra, or a pharmaceutically acceptable salt, tautomer or stereoisomer thereof and at least one therapeutically active agent are administered on the same day. For the simultaneous administration, the compound of formula (I), and/or their subgenra, or a pharmaceutically acceptable salt, tautomer or stereoisomer thereof and at least one therapeutically active agent can be administered at the same time or one at a time.

In one embodiment of the simultaneous administration, the c compound of formula (I), and/or their subgenra, or a pharmaceutically acceptable salt, tautomer or stereoisomer thereof, is administered from 1 to 4 times per day, 1 to 4 times per week, once every two weeks, once every three weeks, once every four weeks or 1 to 4 times per month; and the at least one additional therapeutically active agent is administered 1 to 4 times per day, 1 to 4 times per week, once every two weeks, once every three weeks, once every four weeks or 1 to 4 times per month. In another embodiment of the simultaneous administration, the compound of formula (I), and/or their subgenra, or a pharmaceutically acceptable salt, tautomer or stereoisomer thereof, is administered once a week, once every two weeks, once every three weeks, once every four weeks, or once a month; and the at least one additional therapeutically active agent is administered 1 to 4 times per day, 1 to 4 times per week, once every two weeks, once every three weeks, once every four weeks or 1 to 4 times per month.

By “sequential administration”, it is meant that during a period of two or more days of continuous co-administration without any void day, only one of the compound of formula (I), and/or their subgenra, or a pharmaceutically acceptable salt, tautomer or stereoisomer thereof and at least one therapeutically active agent is administered on any given day.

In one embodiment of the sequential administration, the compound of formula (I), and/or their subgenra, or a pharmaceutically acceptable salt, tautomer or stereoisomer thereof, is administered from 1 to 4 times per day, 1 to 4 times per week, once every two weeks, once every three weeks, once every four weeks or 1 to 4 times per month; and at least one additional therapeutically active agent is administered 1 to 4 times per day, 1 to 4 times per week, once every two weeks, once every three weeks, once every four weeks or 1 to 4 times per month. In another embodiment of the sequential administration, the compound of formula (I), and/or their subgenra, or a pharmaceutically acceptable salt, tautomer or stereoisomer thereof, is administered from once a week, once every two weeks, once every three weeks, once every four weeks, or once a month; and at least one additional therapeutically active agent is administered 1 to 4 times per day, 1 to 4 times per week, once every two weeks, once every three weeks, once every four weeks or 1 to 4 times per month.

By “overlapping administration”, it is meant that during a period of two or more days of continuous co-administration without any void day, there is at least one day of simultaneous administration and at least one day when only one of the compound of formula (I), and/or their subgenra, or a pharmaceutically acceptable salt, tautomer or stereoisomer thereof and at least one therapeutically active agent is administered.

By “interval administration”, it is meant a period of co-administration with at least one void day. By “continuous administration”, it is meant a period of co-administration without any void day. The continuous administration may be simultaneous, sequential, or overlapping, as described above.

In the present method, the co-administration comprises oral administration, parenteral administration, or a combination thereof. Examples of the parenteral administration include, but are not limited to intravenous (IV) administration, intraarterial administration, intramuscular administration, subcutaneous administration, intraosseous administration, intrathecal administration, or a combination thereof. The compound of formula (I), and/or their subgenra, or a pharmaceutically acceptable salt, tautomer or stereoisomer thereof and at least one therapeutically active agent can be independently administered orally or parenterally. In one embodiment, the compound of formula (I), and/or their subgenra, or a pharmaceutically acceptable salt, tautomer or stereoisomer thereof and at least one therapeutically active agent is administered parenterally. The parenteral administration may be conducted via injection or infusion.

In one embodiment of the present method, at least one of the following compounds are provided for use in combination therapy with at least one additional therapeutically active agent:

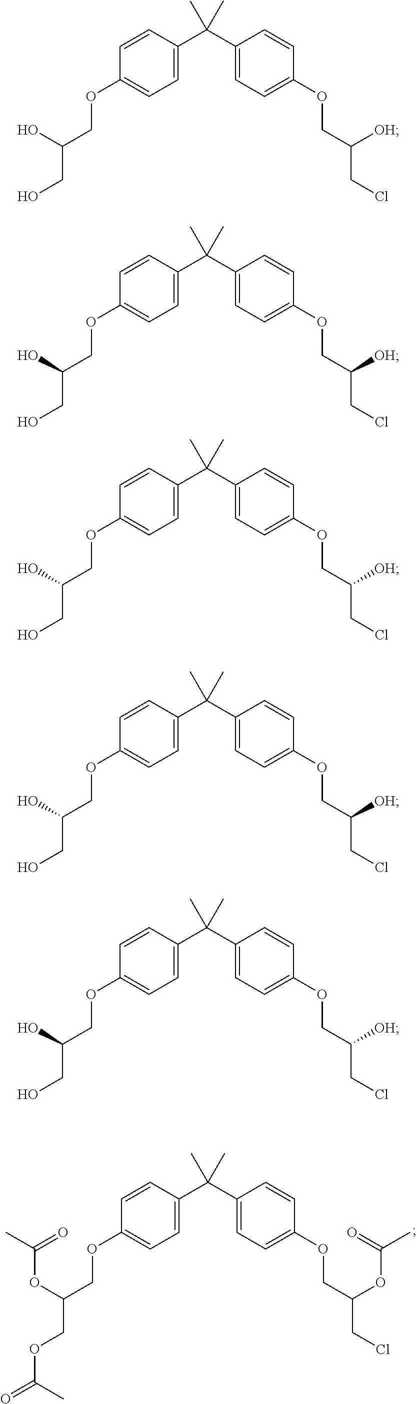 [SMILES: CC(C)(c1ccc(OCC(O)CO)cc1)c1ccc(OCC(O)CCl)cc1 ; CC(C)(c1ccc(OC[C@H](O)CO)cc1)c1ccc(OC[C@@H](O)CCl)cc1 ; CC(C)(c1ccc(OC[C@H](O)CCl)cc1)c1ccc(OC[C@@H](O)CO)cc1 ; CC(C)(c1ccc(OC[C@@H](O)CO)cc1)c1ccc(OC[C@@H](O)CCl)cc1 ; CC(C)(c1ccc(OC[C@H](O)CO)cc1)c1ccc(OC[C@H](O)CCl)cc1 ; CC(=O)OCC(COc1ccc(C(C)(C)c2ccc(OCC(CCl)OC(C)=O)cc2)cc1)OC(C)=O]

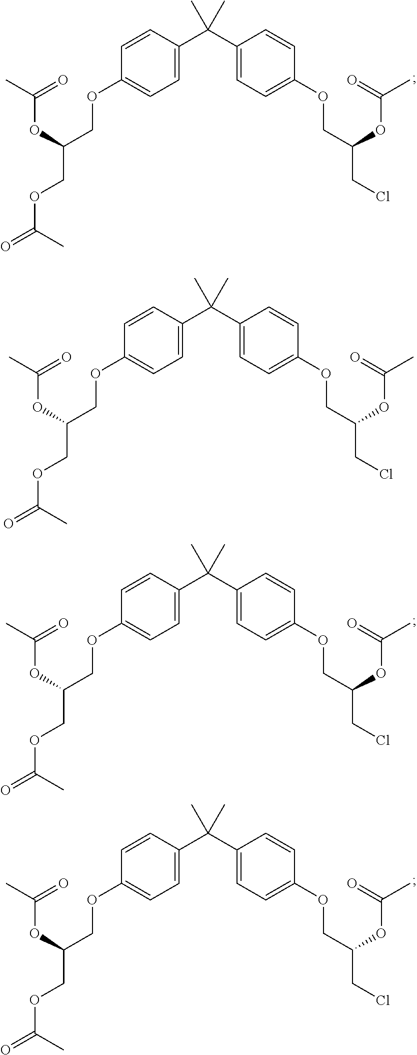

or a pharmaceutically acceptable salt, tautomer, or stereoisomer thereof.

In one embodiment, the at least one additional therapeutically active agent is an inhibitor of the PI3K/AKT/mTOR pathway. In some embodiments, the at least one additional therapeutically active agent is a PI3K/mTOR dual inhibitor. In another embodiment, the at least one additional therapeutically active agent is a PI3K/mTOR dual inhibitor selected from the group consisting of: BEZ-235, XL-765, PF-4691502, GSK-2126458, GDC-0980 and PKI-587. In another embodiment, the PI3K/mTOR dual inhibitor is BEZ-235.

In one embodiment, the compound of formula (I), and/or their subgenra, or a pharmaceutically acceptable salt, tautomer or stereoisomer thereof and at least one additional therapeutically active agent are orally, subcutaneously, or intravenously administered.

### V. Pharmaceutical Formulations

In another embodiment, the present invention provides a pharmaceutical composition and/or combination comprising a therapeutically effective amount of a compound of formula (I), and/or their subgenra, or a pharmaceutically acceptable salt, tautomer or stereoisomer thereof, as disclosed herein, as the active ingredient, combined with a pharmaceutically acceptable excipient or carrier. The excipients are added to the formulation for a variety of purposes.

In some embodiments, the compound of formula (I), and/or their subgenra, or a pharmaceutically acceptable salt, tautomer or stereoisomer thereof and at least one therapeutically active agent may be formulated into a single pharmaceutical composition and/or combination. In some embodiments, the compound of formula (I), and/or their subgenra, or a pharmaceutically acceptable salt, tautomer or stereoisomer thereof and at least one therapeutically active agent are formulated into a separate pharmaceutical composition and/or combination comprising a pharmaceutically acceptable excipient or a carrier.

In one embodiment, the pharmaceutical composition comprising the compound of formula (I), and/or their subgenra, or a pharmaceutically acceptable salt, tautomer or stereoisomer thereof and at least one therapeutically active agent further comprises a second therapeutically active agent or more. The second therapeutically active agent is selected from the group consisting of inhibitors of PI3K/AKT/mTOR pathway, active agents associated with the treatment of prostate cancer, and anticancer agents, disclosed herein.

In one embodiment, the second therapeutically active agent is selected from anticancer agents useful for treating prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, or age-related macular degeneration.

Suitable pharmaceutical compositions can be formulated by means known in the art and their mode of administration and dose determined by the skilled practitioner. Many suitable formulations are known, including, polymeric or protein microparticles encapsulating a compound to be released, ointments, pastes, gels, hydrogels, or solutions which can be used topically or locally to administer a compound. A sustained release patch or implant can be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000).

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

Solid pharmaceutical compositions and/or combinations that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition and/or combination in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition and/or combination, the composition and/or combination is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye.

Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions and/or combinations may also be dyed using any pharmaceutically acceptable colorant to improve their appearance and/or facilitate patient identification of the product and unit dosage level.

In liquid pharmaceutical compositions and/or combinations may be prepared using the compound of formula (I), and/or their subgenra, or a pharmaceutically acceptable salt, tautomer or stereoisomer thereof, of the present invention and any other solid excipients where the components are dissolved or suspended in a liquid carrier such as water, vegetable oil, alcohol, polyethylene glycol, propylene glycol, or glycerin.

Liquid pharmaceutical compositions and/or combinations may contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions and/or combinations may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition and/or combination may also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

The solid compositions and/or combination of the present invention include powders, granulates, aggregates and compacted compositions and/or combinations. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts. For example, a compound can be dissolved in sterile water or saline or a pharmaceutically acceptable vehicle used for administration of non-water soluble compounds such as those used for vitamin K, for a parenteral administration. For enteral administration, the compound can be administered in a tablet, capsule or dissolved in liquid form. The tablet or capsule can be enteric coated, or in a formulation for sustained release.

Dosage forms include solid dosage forms like tablets, powders, capsules, suppositories, sachets, troches and lozenges, as well as liquid syrups, suspensions, aerosols and elixirs.

The dosage form of the present invention may be a capsule containing the composition and/or combination, preferably a powdered or granulated solid composition and/or combination of the invention, within either a hard or soft shell. The shell may be made from gelatin and optionally contain a plasticizer such as glycerin and sorbitol, and an opacifying agent or colorant.

A composition and/or combination for tableting or capsule filling may be prepared by wet granulation. In wet granulation, some or all of the active ingredients and excipients in powder form are blended and then further mixed in the presence of a liquid, typically water that causes the powders to clump into granules. The granulate is screened and/or milled, dried and then screened and/or milled to the desired particle size. The granulate may be tableted, or other excipients may be added prior to tableting, such as a glidant and/or a lubricant.

A tableting composition and/or combination may be prepared conventionally by dry blending. For example, the blended composition and/or combination of the actives and excipients may be compacted into a slug or a sheet and then comminuted into compacted granules. The compacted granules may subsequently be compressed into a tablet.

As an alternative to dry granulation, a blended composition and/or combination may be compressed directly into a compacted dosage form using direct compression techniques. Direct compression produces a more uniform tablet without granules.

Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting; however, they are not subjected to a final tableting step.

The active ingredient and excipients may be formulated into compositions and/or combinations and dosage forms according to methods known in the art.

In one embodiment, a dosage form may be provided as a kit comprising a compound of formula (I), and/or their subgenra, or a pharmaceutically acceptable salt, tautomer or stereoisomer thereof and pharmaceutically acceptable excipients and carriers as separate components. In one embodiment, a dosage form may be provided as a kit comprising a compound of formula (I), and/or their subgenra, or a pharmaceutically acceptable salt, tautomer or stereoisomer thereof at least one additional therapeutically active agent, and pharmaceutically acceptable excipients and carriers as separate components. In some embodiments, the dosage form kit allow physicians and patients to formulate an oral solution or injection solution prior to use by dissolving, suspending, or mixing the compound of formula (I), and/or their subgenra, or a pharmaceutically acceptable salt, tautomer or stereoisomer thereof with pharmaceutically acceptable excipients and carriers. In one embodiment, a dosage form kit which provides a compound of formula (I), and/or their subgenra, or a pharmaceutically acceptable salt, tautomer or stereoisomer thereof which has improved stability when compared to pre-formulated formulations a compound of formula (I), and/or their subgenra, or a pharmaceutically acceptable salt, tautomer or stereoisomer thereof.

In one embodiment, a compound of formula (I), and/or their subgenra, or a pharmaceutically acceptable salt, tautomer or stereoisomer thereof is used in the formulation. The compound of formula (I), and/or their subgenra, or a pharmaceutically acceptable salt, tautomer or stereoisomer thereof, of the present invention may be used in pharmaceutical formulations or compositions and/or combinations as single components or mixtures together with other forms of a compound of formula (I), and/or their subgenra, or a pharmaceutically acceptable salt, tautomer or stereoisomer thereof. In one embodiment, pharmaceutical formulations or compositions and/or combinations of the present invention contain 25-100% or 50-100% by weight, of at least one compound of formula (I), and/or their subgenra, or a pharmaceutically acceptable salt, tautomer or stereoisomer thereof, as described herein, in the formulation or composition and/or combination.

### VI. Therapeutic Use

The present disclosure also provides methods for modulating androgen receptor (AR). Accordingly, in one embodiment, the present disclosure provides the use of any one of the compound of formula (I), and/or their subgenra, or a pharmaceutically acceptable salt, tautomer or stereoisomer thereof, as disclosed herein, for modulating androgen receptor (AR) activity. For example in some embodiments, modulating androgen receptor (AR) activity is in a mammalian cell. Modulating androgen receptor (AR) can be in a subject in need thereof (e.g., a mammalian subject) and for treatment of any of the described conditions or diseases. In one embodiment, the combination of a compound of formula (I), and/or their subgenra, or a pharmaceutically acceptable salt, tautomer or stereoisomer thereof and at least one therapeutically active agent, as disclosed herein, is useful in modulating androgen receptor. In one embodiment, said modulation of AR occurs at the N-terminal domain (NTD).

In other embodiments, modulating androgen receptor (AR) activity is for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof. For example in some embodiments, the indication is prostate cancer. In other embodiments, the prostate cancer is castration resistant prostate cancer (also referred to as hormone refractory, androgen-independent, androgen deprivation resistant, androgen ablation resistant, androgen depletion-independent, castration-recurrent, anti-androgen-recurrent). While in other embodiments, the prostate cancer is androgen dependent prostate cancer. In other embodiments, the spinal and bulbar muscular atrophy is Kennedy's disease.

In one embodiment, the present disclosure provides a method for treating a condition or disease that is responsive to modulation of androgen receptor activity, comprising administering to the subject, a therapeutically effective amount of a compound of formula (I), and/or their subgenra, or a pharmaceutically acceptable salt, tautomer or stereoisomer thereof, as described herein. In one embodiment, the composition of formula (I), and/or their subgenra, or a pharmaceutically acceptable salt, tautomer or stereoisomer thereof and at least one therapeutically active agent, as disclosed herein, is provided in the use of a method for treating conditions or diseases that is responsive to modulation of androgen receptor activity. In some embodiments, said conditions or disease that is responsive to modulation of androgen receptor activity is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, age related macular degeneration, and combinations thereof.

In some embodiments, compounds as described herein can be administered to a subject. In one embodiment, the present invention is directed to a method of treating castration resistant prostate cancer comprising administering a pharmaceutical composition comprising a compound formula (I), and/or their subgenra, or a pharmaceutically acceptable salt, tautomer or stereoisomer thereof and at least one therapeutically active agent, as disclosed herein. In some embodiments, the present invention is directed to a method of treating androgen-dependent prostate cancer comprising administering a pharmaceutical composition comprising a compound formula (I), and/or their subgenra, or a pharmaceutically acceptable salt, tautomer or stereoisomer thereof and at least one therapeutically active agent. In other embodiments, the present invention is directed to a method of treating androgen-independent prostate cancer comprising administering a pharmaceutical composition comprising a compound formula (I), and/or their subgenra, or a pharmaceutically acceptable salt, tautomer or stereoisomer thereof and at least one therapeutically active agent. In one embodiment, the at least one therapeutically active agent is selected from the group consisting of inhibitors of PI3K/AKT/mTOR pathway, active agents associated with the treatment of prostate cancer, and anticancer agents. In one embodiment, the at least one therapeutically active agent is a PI3K/mTOR dual inhibitor.

In other embodiments, the present disclosure provides a method of modulating androgen receptor (AR) activity, the method comprising administering a compound formula (I), and/or their subgenra, or a pharmaceutically acceptable salt, tautomer or stereoisomer thereof, pharmaceutically acceptable salt thereof, or pharmaceutical composition of a compound formula (I), and/or their subgenra, or a pharmaceutically acceptable salt, tautomer or stereoisomer thereof as described herein (including compositions comprising at least one additional therapeutically active agent), to a subject (e.g., mammal) in need thereof. In some embodiments, modulating androgen receptor (AR) activity is in a mammalian cell. In other embodiments, modulating androgen receptor (AR) activity is in a mammal. In one embodiment, modulating androgen receptor (AR) activity is in a human.

The modulating androgen receptor (AR) activity can be for inhibiting AR N-terminal domain activity. The modulating androgen receptor (AR) activity can be for inhibiting androgen receptor (AR) activity. The modulating can be in vivo. The modulating androgen receptor (AR) activity can be for treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy (e.g., Kennedy's disease), and age related macular degeneration. The indication can be prostate cancer. The prostate cancer can be castration-resistant prostate cancer. The prostate cancer can be androgen dependent prostate cancer.

In accordance with another embodiment, there is provided a use of compound formula (I), and/or their subgenra, or a pharmaceutically acceptable salt, tautomer or stereoisomer thereof, and at least one additional therapeutically active agent, as described herein for preparation of a medicament for modulating androgen receptor (AR) or for preparation of a medicament for treatment of cancer, such as prostate cancer and breast cancer.

Alternatively, in one embodiment, a method of modulating androgen receptor activity, comprising administering a compound formula (I), and/or their subgenra, or a pharmaceutically acceptable salt, tautomer or stereoisomer thereof in combination therapy with at least one additional therapeutically active agent. In some embodiments, the administration can be to a mammal. In other embodiments, the administering can be to a mammal in need thereof and in an effective amount for the treatment of at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy (e.g., Kennedy's disease), age related macular degeneration, and combinations thereof.

Androgen ablation therapy causes a temporary reduction in prostate cancer tumor burden, but the malignancy will begin to grow again in the absence of testicular androgens to form castrate resistant prostate cancer (CRPC). A rising titer of serum prostate-specific antigen (PSA) after androgen ablation therapy indicates biochemical failure, the emergence of CRPC, and re-initiation of an androgen receptor (AR) transcription program. Most patients succumb to CRPC within two years of biochemical failure.

AR is a transcription factor and a validated target for prostate cancer therapy. Current therapies include androgen ablation and administration of antiandrogens. Most CRPC is suspected to be AR-dependent. AR has distinct functional domains that include the C-terminus ligand-binding domain (LBD), a DNA-binding domain (DBD), and an amino-terminal domain (NTD). AR NTD contains the activation function-1 (AF-1) that contributes most of the activity to the AR. Recently, splice variants of the AR that lack the LBD have been reported in prostate cancer cell lines (VCaP and 22Rv1), and in CRPC tissues. To date more than 20 splice variants of AR have been detected. Splice variants V7 and V567es are clinically relevant with levels of expression correlated to poor survival and CRPC. AR V567es is solely expressed in 20% of metastases. Abiraterone resistance is associated with expression of AR splice variants. Enzalutamide also increases levels of expression of these constitutively active AR splice variants. These splice variants lack LBD and thereby would not be inhibited by current therapies that target the AR LBD such as antiandrogens or androgen ablation therapy. A single patient with advanced prostate cancer can have many lesions throughout the body and skeleton and each tumor can have differing levels of expression of AR.

In one embodiment, the present disclosure also provides method of treating, reducing, and ameliorating cell proliferation. In one embodiment, the method comprises contacting cancer and/or tumor cells with the compound of formula (I), and/or their subgenra, or a pharmaceutically acceptable salt, tautomer or stereoisomer thereof, as disclosed herein. In another embodiment, the method comprises contacting cancer and/or tumor cells with the compound of formula (I), and/or their subgenra, or a pharmaceutically acceptable salt, tautomer or stereoisomer thereof and at least one therapeutically active agent is administered to the patient in need thereof. Said administration of the compound of formula (I), and/or their subgenra, or a pharmaceutically acceptable salt, tautomer or stereoisomer thereof and at least one therapeutically active agent can be simultaneous administration, sequential administration, overlapping administration, interval administration, continuous administration, or a combination thereof.

In another embodiment, the method of contacting cancer and/or tumor cells with the compound of formula (I), and/or their subgenra, or a pharmaceutically acceptable salt, tautomer or stereoisomer thereof, as disclosed herein, may induce cell apoptosis or alleviate or prevent the progression of the disorder. In one embodiment, the method of contacting cancer and/or tumor cells with the compound of formula (I), and/or their subgenra, or a pharmaceutically acceptable salt, tautomer or stereoisomer thereof and at least one therapeutically active agent, as disclosed herein, may induce cell apoptosis or alleviate or prevent the progression of the disorder.

Additionally, disclosed are methods for treating cancers, cancer cells, tumors, or tumor cells. Non limiting examples of cancer that may be treated by the methods of this disclosure include cancer or cancer cells of: colorectum, breast, ovary, cervix, lung, liver, pancreas, lymph node, colon, prostate, brain, head and neck, skin, kidney, bone (e.g., Ewing's sarcoma) and blood and heart (e.g., leukemia, lymphoma, carcinoma). In one embodiment, the methods of this disclosure include treatment of cancer or cancer cells of prostate or breast cancer. Non limiting examples of tumors that may be treated by the methods of this disclosure include tumors and tumor cells of: colorectum, breast, ovary, cervix, lung, liver, pancreas, lymph node, colon, prostate, brain, head and neck, skin, kidney, bone (e.g., Ewing's sarcoma) and blood and heart (e.g., leukemia, lymphoma, carcinoma). In one embodiment, the methods of this disclosure include treatment of tumors and tumor cells of prostate or breast.

The present invention also provides methods of treating, preventing, ameliorating and/or alleviating the progression of disorders or conditions characterized by cell proliferation in a subject. More particularly, the methods of the present invention involve administration of an effective amount of the compound of formula (I), and/or their subgenra, or a pharmaceutically acceptable salt, tautomer or stereoisomer thereof, in a subject to treat a disorder or a condition characterized by cell proliferation. In one embodiment, the methods of the present disclosure involve administration of an effective amount of the compound of formula (I), and/or their subgenra, or a pharmaceutically acceptable salt, tautomer or stereoisomer thereof and at least one additional therapeutically active agent in a subject to treat a disorder or a condition characterized by cell proliferation.

As used herein, administering can be effected or performed using any of the various methods known to those skilled in the art. The compound of formula (I), and/or their subgenra, or a pharmaceutically acceptable salt, tautomer or stereoisomer thereof, can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles.

Further, the presently disclosed compound of formula (I), and/or their subgenra, or a pharmaceutically acceptable salt, tautomer or stereoisomer thereof, can be administered to a localized area in need of treatment or by means of a medical device or appliances. This can be achieved by, for example, and not by way of limitation, local infusion during surgery, topical application, transdermal patches, by injection, by catheter, by suppository, by implant (the implant can optionally be of a porous, non-porous, or gelatinous material), graft, prosthesis, or stent, including membranes, such as sialastic membranes or fibers.

The form in which the compound of formula (I), and/or their subgenra, or a pharmaceutically acceptable salt, tautomer or stereoisomer thereof, is administered (e.g., syrup, elixir, capsule, tablet, foams, emulsion, gel, etc.) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The compound of formula (I), and/or their subgenra, or a pharmaceutically acceptable salt, tautomer or stereoisomer thereof, can also be used to coat bioimplantable materials to enhance neurite outgrowth, neural survival, or cellular interaction with the implant surface. The compound of formula (I), and/or their subgenra, or a pharmaceutically acceptable salt, tautomer or stereoisomer thereof, disclosed herein can be administered together with other biologically active agents, such as anticancer agents, analgesics, anti-inflammatory agents, anesthetics and other agents which can control one or more symptoms or causes of a disorder or a condition characterized by cell proliferation.

In one embodiment, the compound of formula (I), and/or their subgenra, or a pharmaceutically acceptable salt, tautomer or stereoisomer thereof and additional therapeutically active agent can be administered together with a second therapeutically active agent or more. In one embodiment, the second therapeutically active agent is an anticancer agent. In some embodiments, second therapeutically active agent (or more) is also selected from inhibitors of PI3K/AKT/mTOR pathway, active agents associated with the treatment of prostate cancer, and anticancer agents.

Additionally, administration can comprise administering to the subject a plurality of dosages over a suitable period of time. Such administration regimens can be determined according to routine methods, upon a review of the instant disclosure.

The compound of formula (I), and/or their subgenra, or a pharmaceutically acceptable salt, tautomer or stereoisomer thereof of the invention are generally administered in a dose of about 0.01 mg/kg/dose to about 100 mg/kg/dose. Alternately the dose can be from about 0.1 mg/kg/dose to about 10 mg/kg/dose; or about 1 mg/kg/dose to 10 mg/kg/dose. Time release preparations may be employed or the dose may be administered in as many divided doses as is convenient. When other methods are used (e.g. intravenous administration), the compound of formula (I), and/or their subgenra, or a pharmaceutically acceptable salt, tautomer or stereoisomer thereof, are administered to the affected tissue at a rate from about 0.05 to about 10 mg/kg/hour, alternately from about 0.1 to about 1 mg/kg/hour. Such rates are easily maintained when the compound of formula (I), and/or their subgenra, or a pharmaceutically acceptable salt, tautomer or stereoisomer thereof, are intravenously administered as discussed herein. Generally, topically administered formulations are administered in a dose of about 0.5 mg/kg/dose to about 10 mg/kg/dose range. Alternately, topical formulations are administered at a dose of about 1 mg/kg/dose to about 7.5 mg/kg/dose or even about 1 mg/kg/dose to about 5 mg/kg/dose.

A range of from about 0.1 to about 100 mg/kg is appropriate for a single dose. Continuous administration is appropriate in the range of about 0.05 to about 10 mg/kg.

Drug doses can also be given in milligrams per square meter of body surface area rather than body weight, as this method achieves a good correlation to certain metabolic and excretionary functions. Moreover, body surface area can be used as a common denominator for drug dosage in adults and children as well as in different animal species (Freireich et al., (1966) Cancer Chemother Rep. 50, 219-244). Briefly, to express a mg/kg dose in any given species as the equivalent mg/sq m dose, the dosage is multiplied by the appropriate km factor. In an adult human, 100 mg/kg is equivalent to 100 mg/kg×37 kg/sq m=3700 mg/m².

A dosage form of the present invention may contain a compound of formula (I), and/or their subgenra, or a pharmaceutically acceptable salt, tautomer or stereoisomer thereof, as disclosed herein, in an amount of about 5 mg to about 500 mg. That is, a dosage form of the present invention may contain Compound A in an amount of about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, or 500 mg.

The ratio of the doses of the compound of formula (I), and/or their subgenra, or a pharmaceutically acceptable salt, tautomer or stereoisomer thereof to that of the one or more additional therapeutically active agents can be about 1:1 or can vary, e.g., about 2:1, about 3:1, about 4:1, about 5:1, about 6:1, about 7:1, about 8:1, about 9:1, about 10:1, about 1:2, about 1:3, about 1:4, about 1:5, about 1:6, about 1:7, about 1:8, about 1:9, about 1:10, and can be varied accordingly to achieve the optimal therapeutic benefit.

A dosage form of the present invention may be administered, hourly, daily, weekly, or monthly. The dosage form of the present invention may be administered twice a day or once a day. The dosage form of the present invention may be administered with food or without food.

Insofar as the compound of formula (I), and/or their subgenra, or a pharmaceutically acceptable salt, tautomer or stereoisomer thereof forms disclosed herein can take the form of a mimetic or fragment thereof, it is to be appreciated that the potency, and therefore dosage of an effective amount can vary. However, one skilled in the art can readily assess the potency of the compound of formula (I), and/or their subgenra, or a pharmaceutically acceptable salt, tautomer or stereoisomer thereof of the type presently envisioned by the present application.

In settings of a gradually progressive disorder or condition characterized by cell proliferation, the compound of formula (I), and/or their subgenra, or a pharmaceutically acceptable salt, tautomer or stereoisomer thereof and at least one additional therapeutically active agent are generally administered on an ongoing basis. In certain settings administration of a compound of formula (I), and/or their subgenra, or a pharmaceutically acceptable salt, tautomer or stereoisomer thereof and at least one additional therapeutically active agent disclosed herein can commence prior to the development of disease symptoms as part of a strategy to delay or prevent the disease. In other settings the compound of formula (I), and/or their subgenra, or a pharmaceutically acceptable salt, tautomer or stereoisomer thereof and at least one additional therapeutically active agent disclosed herein is administered after the onset of disease symptoms as part of a strategy to slow or reverse the disease process and/or part of a strategy to improve cellular function and reduce symptoms.

It will be appreciated by one of skill in the art that dosage range will depend on the particular compound of formula (I), and/or their subgenra, or a pharmaceutically acceptable salt, tautomer or stereoisomer thereof and at least one additional therapeutically active agent, and its potency. The dosage range is understood to be large enough to produce the desired effect in which the neurodegenerative or other disorder and the symptoms associated therewith are ameliorated and/or survival of the cells is achieved, but not be so large as to cause unmanageable adverse side effects. It will be understood, however, that the specific dose level for any particular patient will depend on a variety of factors including the activity of the specific compound of formula (I), and/or their subgenra, or a pharmaceutically acceptable salt, tautomer or stereoisomer thereof, employed; the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when Compound A disclosed herein are used in accordance with the present application.

An effective amount of the compound of formula (I), and/or their subgenra, or a pharmaceutically acceptable salt, tautomer or stereoisomer thereof and at least one additional therapeutically active agent disclosed herein comprise amounts sufficient to produce a measurable biological response. Actual dosage levels of active ingredients of the present application can be varied so as to administer an amount of the compound of formula (I), and/or their subgenra, or a pharmaceutically acceptable salt, tautomer or stereoisomer thereof and at least one additional therapeutically active agent that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

Further with respect to the methods of the present application, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the present application indicate effectiveness with respect to all vertebrate species which are included in the term “subject.” In this context, a vertebrate is understood to be any vertebrate species in which treatment of a neurodegenerative disorder is desirable. As used herein, the term “subject” includes both human and animal subjects. Thus, veterinary therapeutic uses are provided in accordance with the present application.

As such, the present application provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos or farms. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided are the treatment of livestock, including, but not limited to, domesticated swine, ruminants, ungulates, horses (including race horses), poultry, and the like.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances, such as in severe disease conditions, substantial excesses of the compositions can be administered for therapeutic effects. Some compounds of this invention can be toxic at some concentrations. Titration studies can be used to determine toxic and non-toxic concentrations. Toxicity can be evaluated by examining a particular compound's or composition's specificity across cell lines using PC3 or DU145 cells as possible negative controls since these cells do not express functional AR. Animal studies can be used to provide an indication if the compound has any effects on other tissues. Systemic therapy that targets the AR will not likely cause major problems to other tissues since antiandrogens and androgen insensitivity syndrome are not fatal.

Compounds for use in the present invention can be obtained from medical sources or modified using known methodologies from naturally occurring compounds. In addition, methods of preparing or synthesizing compounds of the present invention will be understood by a person of skill in the art having reference to known chemical synthesis principles. For example, Auzou et al 1974 European Journal of Medicinal Chemistry 9(5), 548-554 describes suitable synthetic procedures that can be considered and suitably adapted for preparing compounds of any one of the compounds of structure (I) as set out above. Other references that can be helpful include: Debasish Das, Jyh-Fu Lee and Soofin Cheng “Sulfonic acid functionalized mesoporous MCM-41 silica as a convenient catalyst for Bisphenol-A synthesis” Chemical Communications, (2001) 2178-2179; U.S. Pat. No. 2,571,217 Davis, Orris L.; Knight, Horace S.; Skinner, John R. (Shell Development Co.) “Halohydrin ethers of phenols.” (1951); and Rokicki, G.; Pawlicki, J.; Kuran, W. “Reactions of 4-chloromethyl-1,3-dioxolan-2-one with phenols as a new route to polyols and cyclic carbonates.” Journal fuer Praktische Chemie (Leipzig) (1985) 327, 718-722.

In some embodiments, compounds and all different forms thereof as described herein can be used, for example, and without limitation, in combination with other treatment methods for at least one indication selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age related macular degeneration. For example, compounds and all their different forms as described herein can be used as neoadjuvant (prior), adjunctive (during), and/or adjuvant (after) therapy with surgery, radiation (brachytherapy or external beam), or other therapies (eg. HIFU), and in combination with chemotherapies, androgen ablation, antiandrogens or any other therapeutic approach.

The compounds described herein can be used for in vivo or in vitro research uses (i.e. non-clinical) to investigate the mechanisms of orphan and nuclear receptors (including steroid receptors such as androgen receptor (AR)). Furthermore, these compounds can be used individually or as part of a kit for in vivo or in vitro research to investigate signal transduction pathways and/or the activation of orphan and nuclear receptors using recombinant proteins, cells maintained in culture, and/or animal models.

### EXAMPLES

### Material and Methods

Cells, Reporter Assays and Reagents:

LNCaP and Cos-1 cells, plasmids (PSA-luciferase, PB-luciferase, ARR3-luciferase, 5xGal4UAS-TATA-luciferase, AR1-558Gal4DBD) and transfection protocols have been described previously(7, 12). LNCaP95 cells from Dr. Stephen R. Plymate (University of Washington, Seattle, Wash.) is an androgen-independent cell line, that expresses FL-AR and constitutively active AR-V7 which lacks the ligand-binding domain. Compound A was provided by NAEJA (Edmonton, Alberta). Enzalutamide was purchased from Omega Chem (St-Romuald, Quebec). NVP-BEZ235 was purchased from SelleckChem (Boston, Mass.). The synthetic androgen, R1881, was purchased from Perkin-Elmer (Woodbridge, ON). Interleukin-6 was from R&D Systems (Minneapolis, Minn.). Forskolin was from EMD Millipore (Billerica, Mass.). Silencer select siRNA for p110 beta (# s10523, 10524 and 10525), p110 gamma (#s 10529, 10530 and 10531), and lipofectamine RNAiMAX were from Life Technologies (Carlsbad, Calif.). All cells were maintained in culture no more than 10 passages and regularly tested to ensure they were mycoplasma-free.

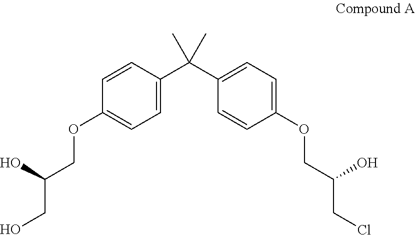 [SMILES: CC(C)(c1ccc(OC[C@H](O)CO)cc1)c1ccc(OC[C@H](O)CCl)cc1]

Cell Proliferation BrdU Immunoassay:

LNCaP95 cells (8,000 cells/well) were seeded in a 96-well plate and incubated for 48 h in RPMI with 10% charcoal stripped serum before pre-treating for 1 h with DMSO, Compound A (25 uM), enzalutamide (10 uM), BEZ235 (15 nM) and combination of Compound A (25 uM) and BEZ235 (15 nM) in serum-free conditions prior to addition of 0.1 nM R1881 or EtOH. BrdU incorporation was measured after 2 days, using BrdU ELISA kit (Roche Diagnostics) according to the manufacturer's protocol.

Western Blot Analysis:

LNCaP95 cells (250,000 cells/well) were seeded in a 6-well plate for 48 hr, and serum-starved for 24 hr, followed by treatment with DMSO, Compound A (25 uM), enzalutamide (10 uM), BEZ235 (15 nM) or a combination for 1 h prior to addition of R1881 or EtOH for 48 hr. Cells were harvested and whole-cell lysate (10 to 15 ug) was subjected to SDS-PAGE. Antibodies used were: AR (1:1000; Santa Cruz), AR-V7 (1:400; Precision), p110α (1:500; BD Bioscience), p110β (1:1000; abcam), p100γ (1:1000; abcam), p110δ (1:1000; abcam), UBE2C (Boston Biochem; 1:1000), PTEN (1:1000), pS6 (1:2000), pAktThr308 (1:1000), pAktSer473 (1:2000), p4EBP1 (1:1000), total-Akt (1:1000), total-S6 (1:1000), total-4EBP1 (1:1000), pERK/MAPK (1:1000), total-ERK/MAPK (1:1000) from Cell Signaling technology (Danvers, Mass.). β-actin (1:10,000, Abcam) was used as a loading control.

Gene Expression Analysis:

LNCaP95 cells (180,000 cells/well) in a 6-well plate were serum-starved for 24 h before treating with vehicle, Compound A (35 uM), enzalutamide (10 uM), BEZ235 (15 nM) or its combination for 1 h prior to the addition of R1881 (1 nM) or EtOH for 48 h. Total RNA was isolated using PureLink RNA Mini Kit (Life technologies) and reverse transcribed to cDNA with High Capacity RNA-to-DNA Kit (Life Technologies). Quantitative real-time RT-PCR was performed in triplicates for each biological sample. Expression levels were normalized to RPL13A housekeeping gene. Primers are as previously described (Andersen, R. J. et al. Cancer Cell 2010, 17, 535-546; Zhang, X. et al. PloS one. 2011, 6, e27970).

Animal Studies:

Six to eight weeks old male NOD-SCID mice were maintained in the Animal Care Facility in the British Columbia Cancer Research Centre. All animal experiments were approved by the University of British Columbia Animal Care Committee. Mice were castrated two weeks before inoculating LNCaP95 cells (5 million cells/tumor) subcutaneously, and divided into 4 groups: vehicle control (N-Methyl-2-pyrrolidone:polyethylene glycol 400 (10/90, v/v), n=10), Compound A (100 mg/kg bodyweight, n=8), BEZ235 (5 mg/kg body weight, n=10) and combination of Compound A (100 mg/kg body weight) and BEZ235 (5 mg/kg body weight) (n=8). Solutions were prepared fresh every day and application volume was 5 ml/kg body weight/dose. Animals were treated by oral gavage, qd, for 2 weeks when tumors reached approximately 80 mm³. Body weight was measured every day and tumor volumes were measured twice a week using a caliper by the formula length×width×height×0.52. Tumors were harvested 1 h after the last treatment and prepared for western blot analyses, gene expression assays and immunohistochemistry.

Immunohistochemistry:

For immunohistochemical staining, sections (5 um thick) were cut from formalin fixed paraffin-embedded tissues and deparaffinized in xylene and rehydrated in alcohols and distilled water. Endogenous peroxidase was blocked with 3% hydrogen peroxide in distilled water for 5 min, followed by washing in PBST three times. Sections were then incubated with super blocking buffer for 30 min to prevent the non-specific bindings of antibodies and then with anti-pS6 (1:200; cell signaling), anti-UBE2C (1:200; Boston Biochem) and anti-Ki-67 (1:50; Dako) at 4° C. overnight. This was followed by incubation with biotinylated secondary antibodies for 30 min and avidin-biotin peroxidase complex for 30 min at room temperature. Antigen was detected with 3,3-diaminobenzidine and counterstaining with hematoxylin. For TUNEL staining, ApopTag® Fluorescein In Situ Apoptosis Detection Kit (MILLIPORE) was used.

### Example 1

### Determination of the Effect of Co-Targeting AR-NTD and mTOR with Compound a and Low Dose BEZ-235 or Everolimus

LNCaP95 human prostate cancer cells are androgen-independent and enzalutamide-resistant (Hu, R. et al., Cancer Research 2012, 72, 3457-3462; Yang, Y. C. et al., Molecular Cancer Therapeutics 2013, 12, 621-631). The proliferation of LNCaP95 cells is driven by truncated AR splice variant (AR-Vs) in spite of endogenous expression of functional full-length AR (FL-AR). Compound A is an antagonist of AR activation function 1 (AF-1) that blocks the activity of both full-length and truncated AR species (Andersen R. J. et al., Cancer Cell 2010, 17, 535-546; Myung J. K. et al., J. Clin. Invest. 2013, 123, 2948-2960; Yang, Y. C. et al., Molecular Cancer Therapeutics 2013, 12, 621-631).

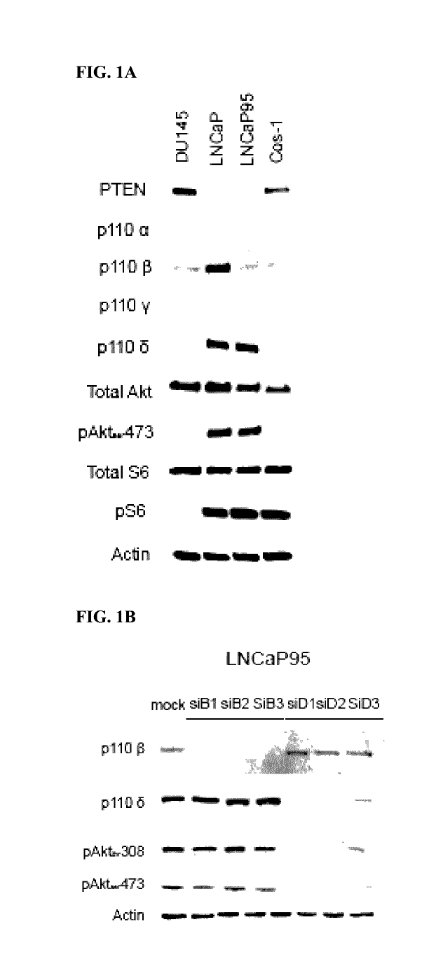

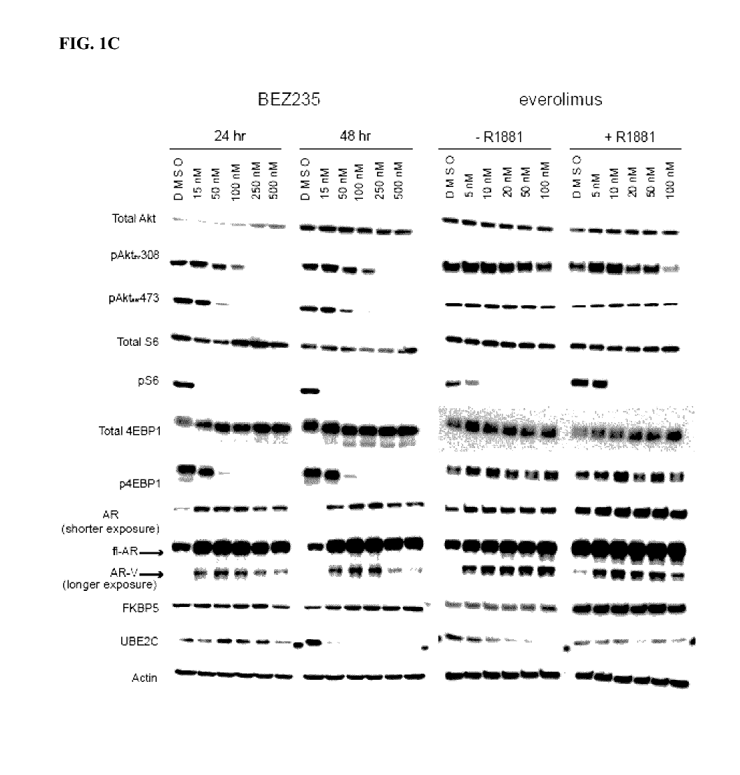

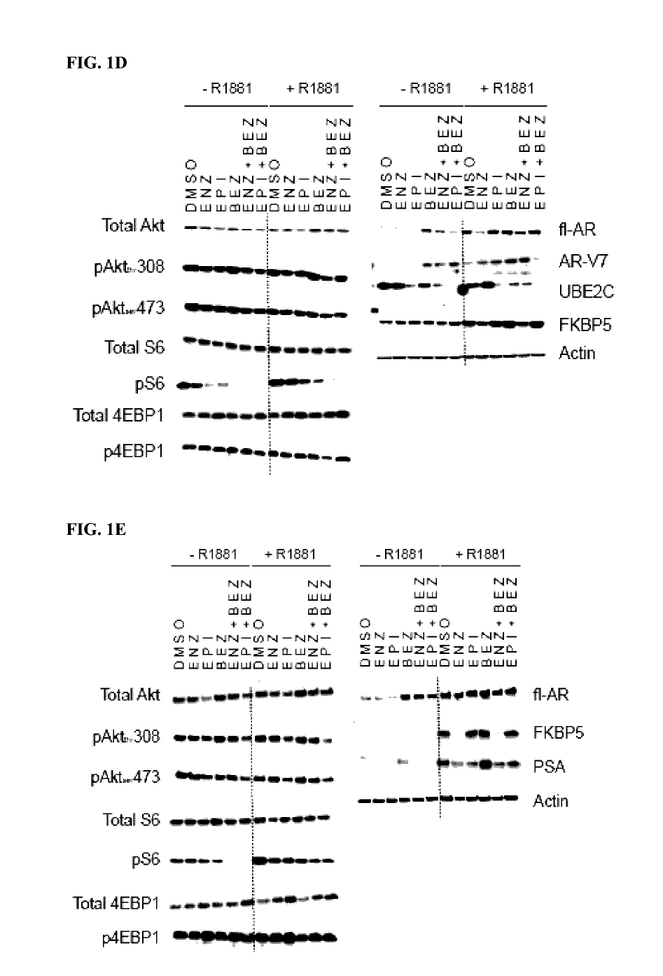

To determine the functional roles of FL-AR, AR-Vs and PI3K/Akt/mTOR pathways, Compound A (EPI) or enzalutamide (ENZ) and BEZ-235 (BEZ) or everolimus were employed in human prostate cancer cells that express FL-AR or FL-AR and AR-Vs (LNCaP95). Comparative expression levels of p110 isoforms, pAkt, and pS6 (phosphorylation of S6) were evaluated in cell lines (FIG. 1A). LNCaP and LNCaP95 cells are PTEN null and express p1106 and p110(3 isoforms albeit LNCaP95 have much lower levels of p110(3 than LNCaP.

Using siRNA to p1106 or p1103, revealed that phosphorylation of Akt (pAkt) in LNCaP95 cells depends predominantly upon p1106 (FIG. 1B). This was determined by knockdown of p110 β (siB1,2,3) or p¹¹⁰ 6 (siD1,2,3) in LNCaP95 cells for 48 h followed by analyses of levels of pAkt were measured. BEZ-235 is a dual PI3K/mTOR inhibitor and in cell-free assays has the following IC50s in the nM range: p110α, 4 nM; mTOR (p70S6K), 6 nM; p1106, 7 nM; ATR, 21 nM; and p110β, 75 nM (Maria, S. M. et. al., Molecular Cancer Therapeutics 2008, 7, 1851-1863; Chiarini, F. et al., Cancer Research 2010, 70, 8097-8107). At higher concentrations BEZ-235 inhibits EGFR/Erb1>8.5 uM and many more kinases at >10 uM including Akt1, IGF1R, and CDK1 (Maria, S. M. et. al., Molecular Cancer Therapeutics 2008, 7, 1851-1863). However, the previously reported concentration of 500 nM BEZ-235 that was used to inhibit pAkt in LNCaP cells (Carver, B. S. et al., Cancer Cell 2011, 19, 575-586) also inhibited pAkt here in LNCaP95 cells but was associated with enormous cytotoxicity making it difficult to interpret the data.

Titration experiments revealed a non-toxic concentration of 15 nM BEZ-235 that was subsequently used in all experiments, but this concentration did not impact pAkt (FIG. 1C). LNCaP95 cells were exposed for 24 h or 48 h to BEZ-235 or 24 h to everolimus at various concentrations. In LNCaP95 cells, BEZ-235 (15 nM) inhibited pS6 ribosomal protein, an mTOR-regulated protein but not p4EBP1 levels. Consistent with previous reports, BEZ-235 increased protein levels of FL-AR but unexpected were the decreased levels of UBE2C at 48 h which is an ARV7 target gene (FIG. 1C). Everolimus, an mTOR inhibitor, reduced pS6 at 10 nM and in the absence of androgen also reduced levels of UBE2C.

Combination experiments with BEZ-235 (15 nM) were examined in LNCaP95 cells compared to the parental LNCaP cells. Compound A reduced pS6 levels regardless of androgen status (FIG. 1D). In the absence of androgen, BEZ-235 increased levels of FL-AR and AR-V7. Compound A, but not enzalutamide, also markedly reduced expression of UBE2C, consistent with previous reports (Myung J. K. et al., J. Clin. Invest. 2013, 123, 2948-2960; Yang, Y. C. et al., Molecular Cancer Therapeutics 2013, 12, 621-631).

Effects of enzalutamide, Compound A and BEZ-235 on mTOR and AR pathways in LNCaP95 were determined using LNCaP95 cells which were serum-starved for 24 h and then treated with DMSO, Compound A (25 uM), enzalutamide (10 uM), BEZ-235 (15 nM) or combination for 1 h prior to the addition of R1881 (1 nM) or EtOH for 48 h. In the presence of androgen, Compound A had no effect on levels of FKBP5, a gene transcriptionally regulated by FL-AR (FIG. 1D). Protein levels of PSA were undetectable in LNCaP95 cells. LNCaP cells do not express constitutively active AR splice variant and, are androgen sensitive with proliferation dependent on AR. No studies have been reported using a concentration of 15 nM BEZ-235 in LNCaP cells. BEZ-235 had no effect on pAKT at this concentration.

Effects of enzalutamide, Compound A and BEZ-235 on mTOR and AR pathways in PARENTAL LNCAP were determined using parental LNCaP cells which were serum-starved for 24 h and then treated with DMSO, Compound A (25 uM), enzalutamide (10 uM), BEZ-235 (15 nM) or combination for 1 h prior to the addition of R1881 (1 nM) or EtOH for 48 h. Combinations of BEZ-235 with enzalutamide or Compound A were substantially better than BEZ-235 monotherapy in blocking pS6 (FIG. 1E). In the absence of androgen, BEZ-235 increased levels of FL-AR (FIG. 1E). Consistent with results obtained with LNCaP95 cells, Compound A was a poor inhibitor of androgen-induced FKBP5 in spite of being comparable to enzalutamide in blocking androgen-induced levels of PSA (FIG. 1E). PSA was increased with BEZ-235 regardless of androgen status. Thus, although BEZ-235 increased protein levels of FL-AR, AR-V7, and possibly downstream target genes (PSA), these elevations were at least in part attenuated by Compound A and enzalutamide. In summary, Compound A inhibited AR-V7 and mTOR and also blocked BEZ-235-induced FL-AR transcriptional activity.

### Example 2

### Determination of the Effect of Inhibition of mTOR on AR Transcription Activity

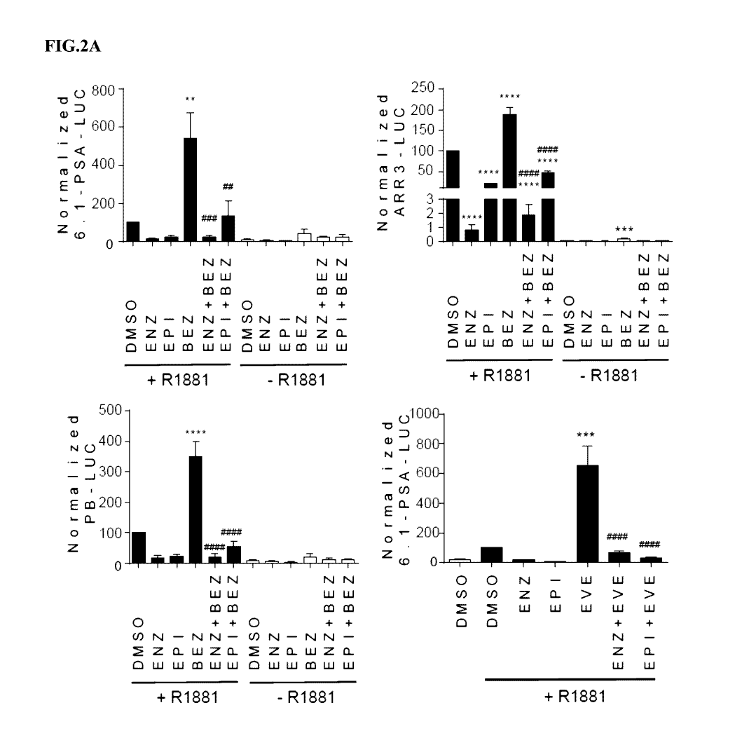

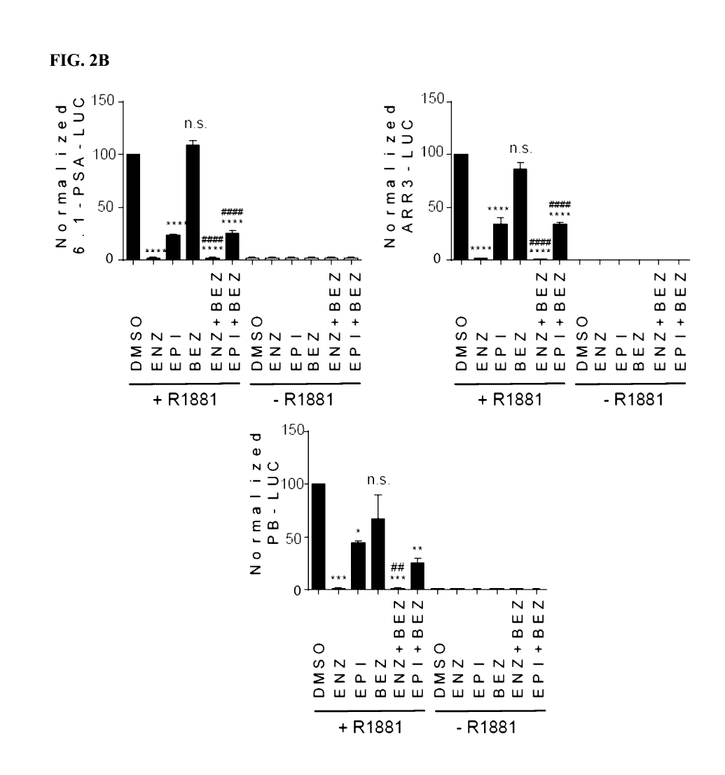

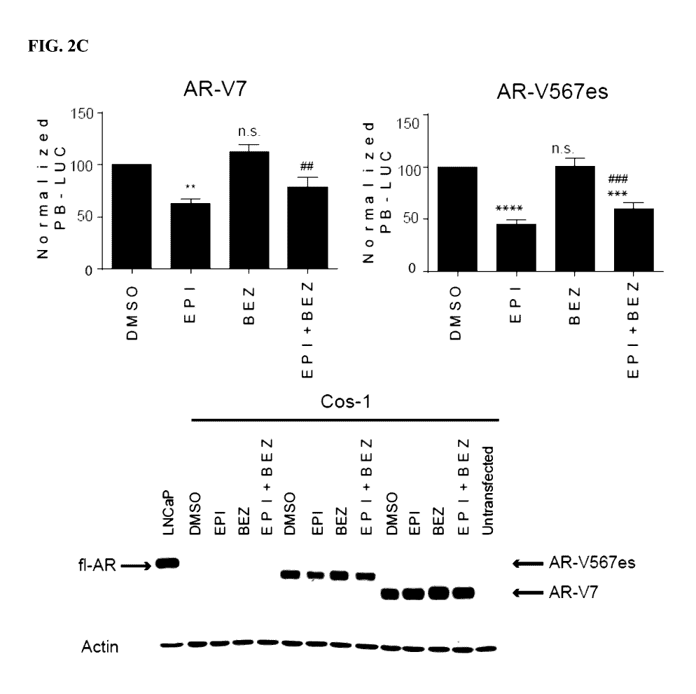

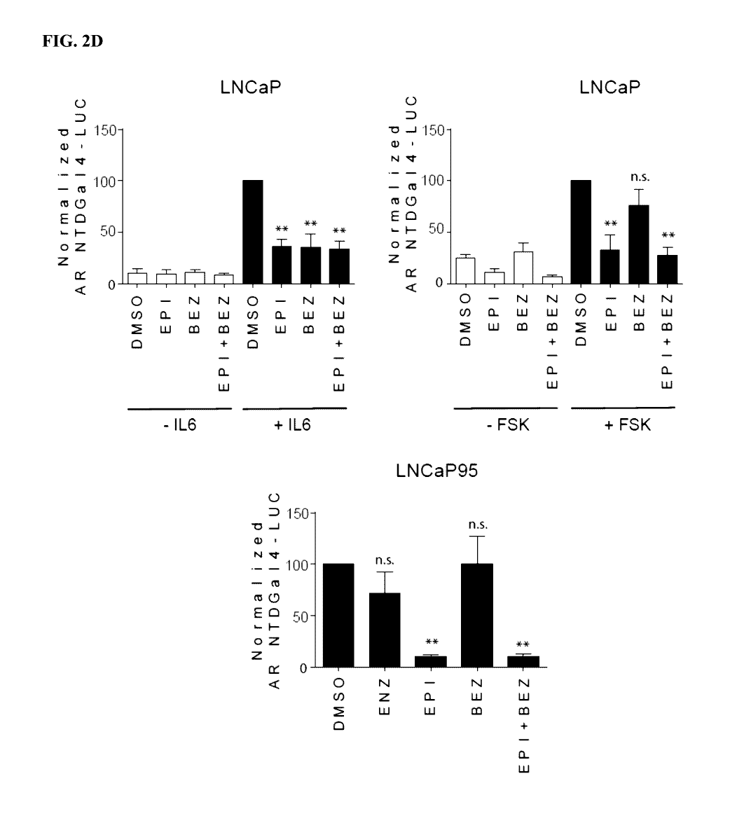

PSA-, ARR3- and PB-luciferase are three well-characterized androgen-induced AR-driven reporter gene constructs. LNCaP95 and LNCaP cells transiently transfected with PSA-, ARR3- or PB-luciferase reporters were treated with DMSO, Compound A (EPI), enzalutamide (ENZ), BEZ-235 (BEZ) or combinations thereof for 1 hr prior to the addition of R1881 for 48 h in serum-free conditions. LNCaP95 cells transfected with PSA-luciferase reporter were also treated with everolimus (10 nM) or combination with enzalutamide or Compound A to compare with results using BEZ-235 (FIGS. 2A and 2B). BEZ-235 (15 nM) significantly increased PSA-, ARR3- and PB-luciferase activities in LNCaP95 cells treated with androgen which were blocked by both enzalutamide and Compound A (FIG. 2A). To confirm this change was through inhibition of mTOR, LNCaP95 cells were treated with everolimus (EVE, 10 nM) which yielded a similar increase in PSA-luciferase activity (FIG. 2A). Importantly, in LNCaP cells BEZ-235 did not enhance the activity of FL-AR in response to androgen (FIG. 2B).

Next, to address whether BEZ-235 affected AR-Vs transcriptional activities, Cos-1 cells that do not express endogenous AR were transiently co-transfected with PB-luciferase and expression vectors for AR-V567 or AR-V7 for 5 h, and then treated with DMSO, Compound A, BEZ-235 or combination of Compound A and BEZ-235 for 24 h in serum-free conditions prior to measuring luciferase activities (FIG. 2C). BEZ-235 had no effect on the transcriptional activities of either AR-V567 or AR-V7 in Cos-1 cells. Ectopic protein levels of AR-V7 and AR-V567 in Cos-1 cells are shown relative to endogenous levels of FL-AR in LNCaP cells. Protein levels of AR-V7 and Ar-567 were comparable to endogenous levels in LNCaP cells (FIG. 2C).

To determine if BEZ-235 directly enhanced AR transactivation, the AR NTD transactivation assay using both LNCaP and LNCaP95 cells were employed. Transactivation assays of the AR NTD were performed in LNCaP and LNCaP95 cells cotransfected with p5xGal4UAS-TATA-luciferase and AR NTD-Gal4DBD (FIG. 2D). Compound A, BEZ-235, or combination of Compound A and BEZ-235 were added 1 h before addition of IL-6 (50 ng/ml) or forskolin (FSK; 50 uM) in LNCaP cells and harvested after 24 h. LNCaP95 cells were harvested 24 h after the treatment of indicated compounds.

The AR NTD is essential for full transcriptional activities (Quayle, S. N. et. al., PNAS 2007, 104, 1331-1336). In LNCaP cells, transactivation of the AR NTD can be induced with IL-6 or by stimulation of the PKA pathway with FSK. In LNCaP95 cells, there is high intrinsic activity of the AR NTD which cannot be further induced by stimulation of these pathways. In LNCaP cells, BEZ-235 as well as Compound A (positive control) significantly inhibited AR-NTD transactivation induced by IL-6 (FIG. 2D). BEZ-235 had no effect on AR-NTD transactivation induced by FSK in LNCaP cells or on the intrinsic activity of AR NTD in LNCaP95. Taken together, BEZ-235 has differential effects on AR transcriptional activities that possibly involve cell-specific differences in signal transduction pathways. In summary, BEZ-235 increased FL-AR transcriptional activity in LNCaP95 and inhibited IL-6 induced transactivation in LNCaP, but had no effect on ectopic AR-V567 and AR-V7 transcriptional activities in Cos-1 cells.

Luciferase activities were shown as percentage of vehicle control. Data in FIGS. 2A-2D is presented as the mean±SEM from three independent experiments. One-Way ANOVA, post-hoc Turkey's multiple comparisons test. * indicate vs DMSO control. # indicate vs BEZ-235 treatment group. n.s.; not statistically significant; *p<0.05; ** p<0.01; *** p<0.001; ****p<0.0001; ## p<0.01; ### p<0.001; #### p<0.0001.

### Example 3

### Determination of the Effect of Compound A and BEZ-235 on Endogenous Genes Regulated by FL-AR and AR-V7

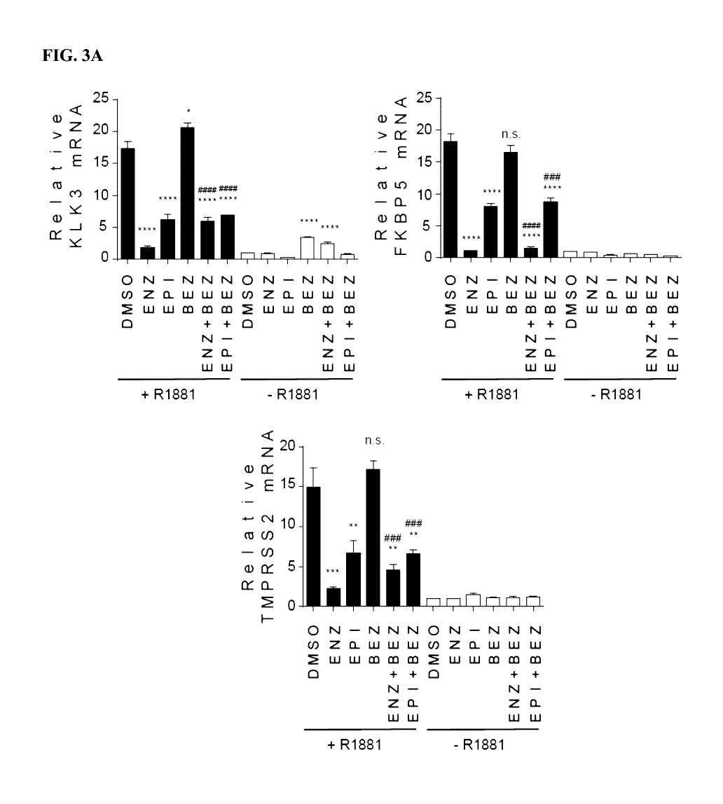

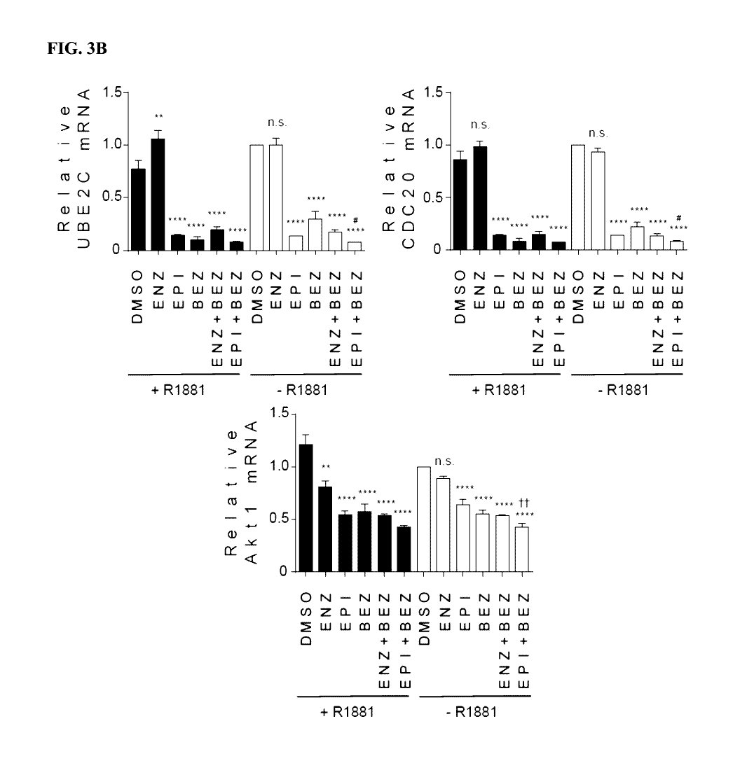

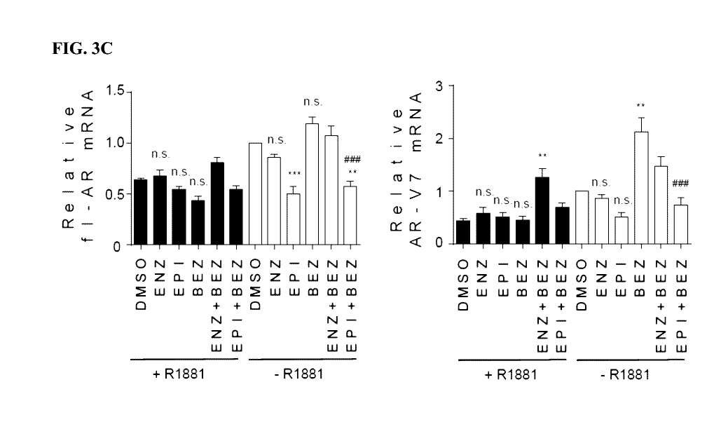

LNCaP95 cells were next tested to examine the effects of BEZ-235 and combination therapies on endogenous gene expression regulated by FL-AR and AR-Vs. LNCaP95 cells were serum-starved for 24 h and then treated with DMSO, Compound A (EPI; 35 uM), enzalutamide (ENZ), BEZ-235 (BEZ) or combination of enzalutamide and BEZ-235 or Compound A and BEZ-235 for 1 h prior to the addition of R1881 or EtOH for 48 h. Compound A and enzalutamide inhibited expression of KLK3, TMPRSS2 and FKBP5, which are genes regulated by FL-AR in response to androgen. Importantly, BEZ-235 significantly increased androgen-induced levels of PSA transcripts compared to levels induced by androgen alone (FIG. 3A). In the absence of androgen, BEZ-235 also induced levels of PSA transcript which could be blocked by Compound A but not enzalutamide. No similar effects were observed for TMPRSS2 or FKBP5 in response to BEZ-235.

AR-V7 regulates a subset of genes that are unique from FL-AR. Enzalutamide increased levels of UBE2C transcripts in cells treated with androgen, while monotherapy with Compound A or BEZ-235 attenuated UBE2C levels regardless of androgen (FIG. 3B). In the absence of androgen, enzalutamide had no effect on transcript levels of any of the AR-V7 target genes, contrary to monotherapies with Compound A or BEZ-235 that consistently reduced levels of expression of these AR-V7 target genes. The combination of Compound A and BEZ-235 were significantly more effective than monotherapies. BEZ-235 did not increase levels of FL-AR transcript (FIG. 3C). Surprising was the greater than 2-fold increase in transcript levels of AR-V7 induced with BEZ-235 in the absence of androgen which was blocked by Compound A (FIG. 3C). In summary, Compound A inhibited both FL-AR and AR-V7 regulated genes, while BEZ-235 inhibited AR-V7 regulated genes.

In FIGS. 3A-3C, all transcripts were normalized to levels of RPL13A. Error bars represent the mean±SEM from three independent experiments. One-Way ANOVA, post-hoc Turkey's multiple comparisons test. * indicate vs DMSO control. † indicate vs EPI treatment group. # indicate vs BEZ treatment group. n.s.; not statistically significant; *p<0.05; ** p<0.01; *** p<0.001; ****p<0.0001; t p^{<}0.01; # p<0.05; ### p<0.001; #### p<0.0001.

### Example 4

### Determination of the Effect of a Combination Therapy with Compound A and BEZ-235 on Tumor Growth

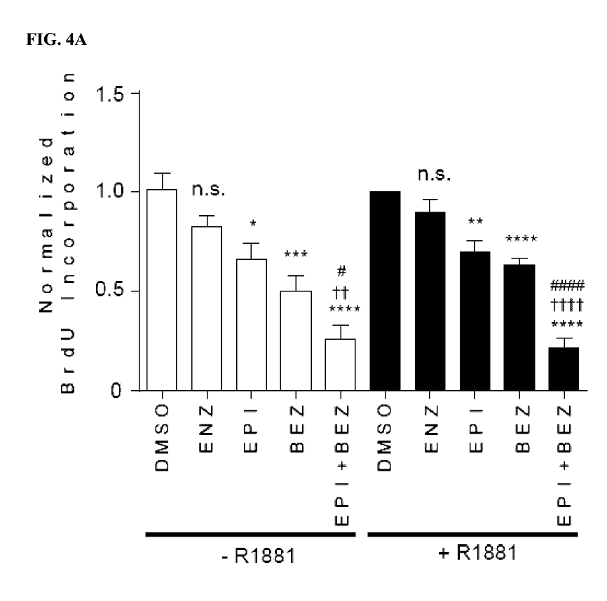

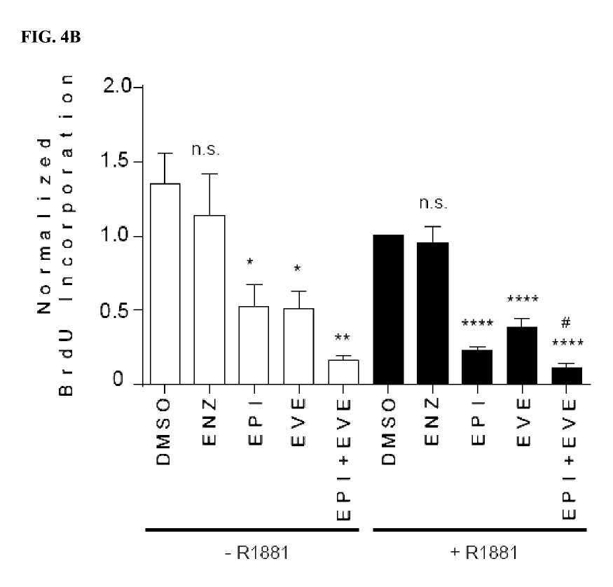

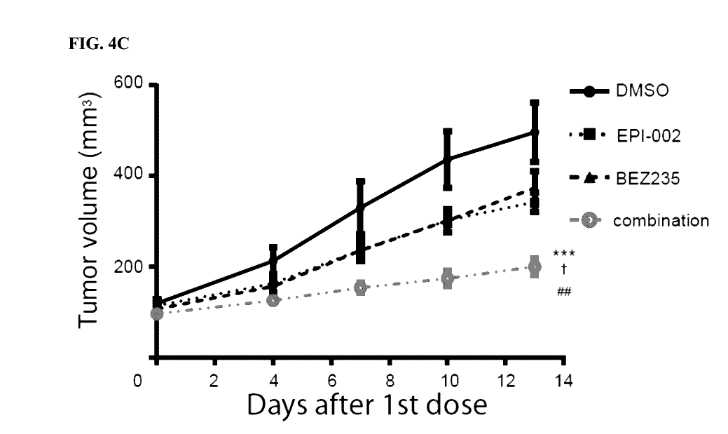

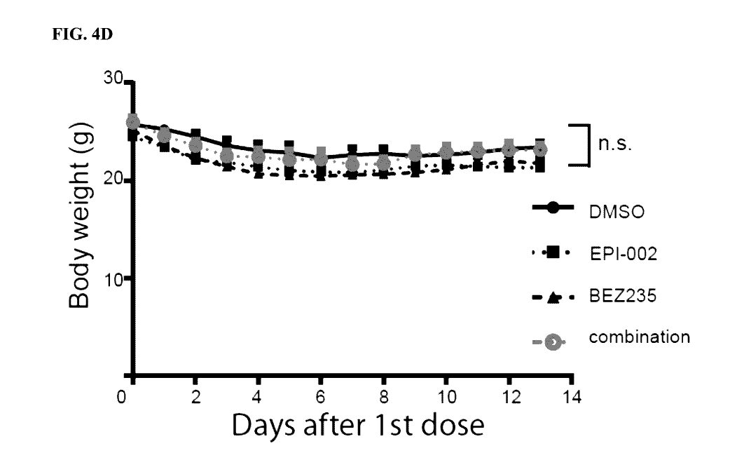

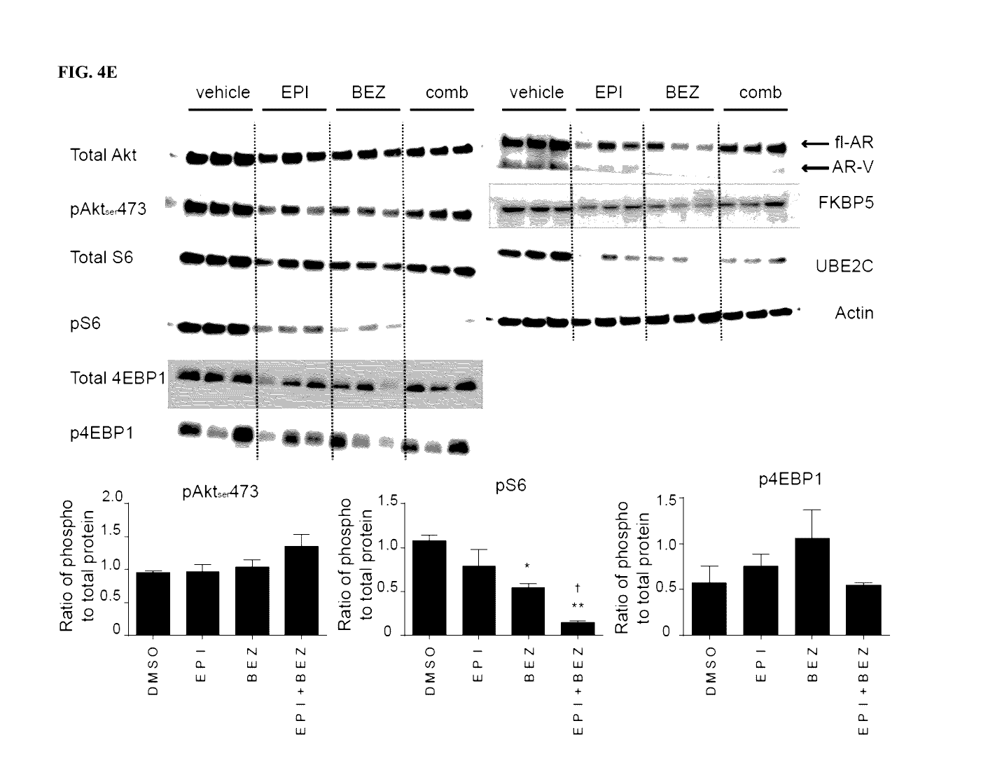

Proliferation of LNCaP95 cells is androgen-independent and driven by AR splice variant (Hu, R. et al., Cancer Research 2012, 72, 3457-3462; Hu, R. et al., Cancer Research 2009, 69, 16-22). LNCaP95 cells were treated with DMSO, Compound A (EPI), enzalutamide (ENZ), BEZ-235 (BEZ) or combination of Compound A and BEZ-235 for 1 h prior to the addition of R1881 (0.1 nM) for 48 h in serum-free media. Proliferation was measured by BrdU incorporation. As expected, enzalutamide had no effect on the proliferation of these cells (FIG. 4A). Thus, LNCaP95 cells are enzalutamide-resistant.

Compound A or BEZ-235 monotherapies inhibited proliferation with the combination being significantly better than each monotherapy. LNCaP95 cells were also treated with everolimus (EVE, 10 nM) instead with BEZ-235. Everolimus also inhibited the proliferation of LNCaP95 cells, indicating that this additional inhibition was accomplished through mTOR inhibition (FIG. 4B). Everolimus in combination with Compound A was significantly better than the monotherapies.

A small pilot in vivo study was completed to determine the non-toxic oral dose of BEZ-235 that could be administered daily. Doses of BEZ-235 at 45 mg/kg body weight resulted in the mortality of 66% of the animals. BEZ-235 orally administered daily at 5 mg/kg body weight was non-toxic and sufficient to block mTOR but not pAkt in tumors. Therefore a dose of BEZ-235 at 5 mg/kg body weight was used in the following in vivo studies.

Castrated mice were daily treated orally either with vehicle (NMP:PEG400, 1:9, v/v), a half-dose of Compound A (100 mg/kg), BEZ-235 (5 mg/kg) or a combination (Compound A 100 mg/kg+BEZ-235 5 mg/kg) for two weeks. The final tumor volume in the Compound A+BEZ-235 combination group was significantly reduced compared to those in vehicles (DMSO), Compound A and BEZ-235 groups (FIG. 4C). There was no significant difference in body weight among the treatment groups (FIG. 4D). Interestingly, protein levels of FL-AR and AR-V7, in response to BEZ-235 were reduced contrary to in vitro results that showed an increase in FL-AR and AR-V7 protein levels, as determined by Western blot analyses of protein lysates from xenografts harvested 1 h after the last treatment (FIG. 4E). Consistent with in vitro data, protein levels of UBE2C and pS6 were reduced in harvested tumors treated with Compound A, BEZ-235 and combination treatment, but no significant change was observed in levels of pAkt and p4EBP1 when normalized to total Akt or 4EBP1, respectively (FIG. 4E).

In FIGS. 4A-4E, error bars represent the mean±SEM from at least three independent experiments. One-Way ANOVA, post-hoc Turkey's multiple comparisons test. * indicate vs DMSO control. † indicate vs Compound A treatment group. # indicate vs BEZ-235 treatment group. n.s.; not statistically significant; *p<0.05; ** p<0.01; *** p<0.001; ****p<0.0001; † p^{<}0.05; †† p^{<}0.01; †††† p^{<}0.0001; # p<0.05; ## p<0.01; #### p<0.0001.

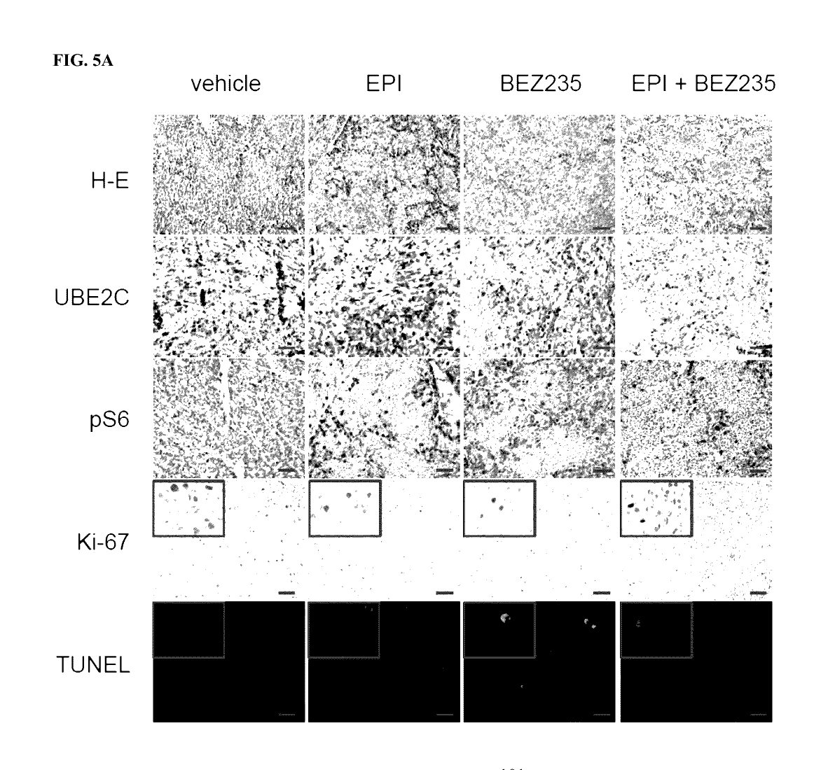

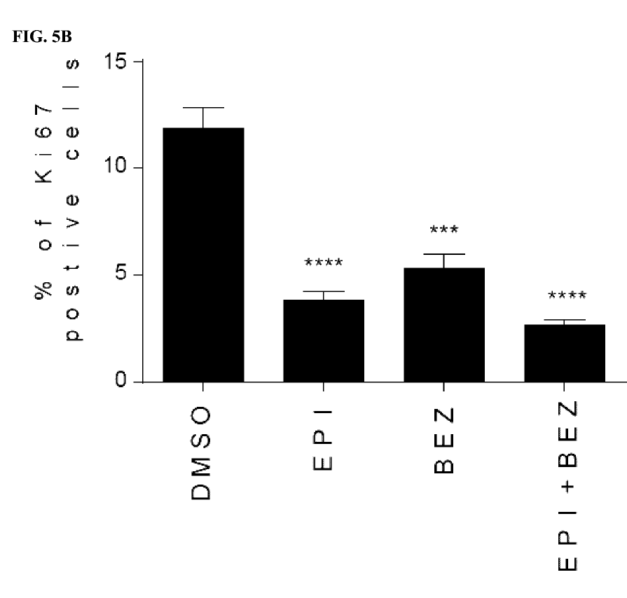

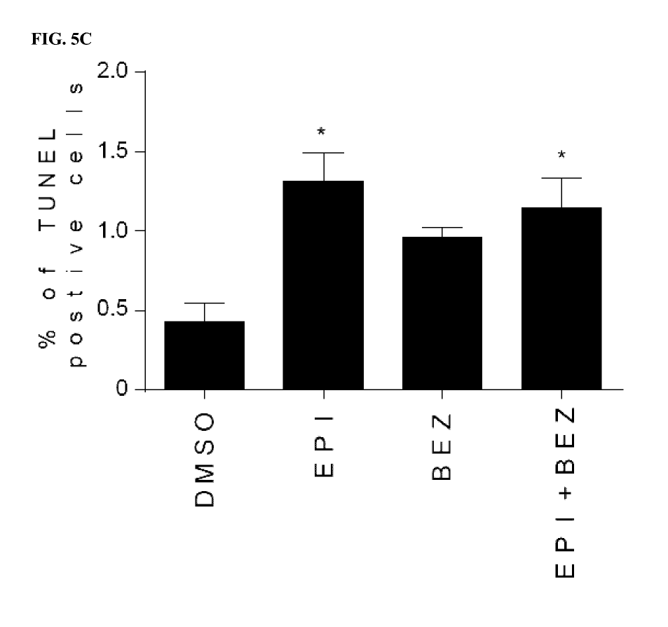

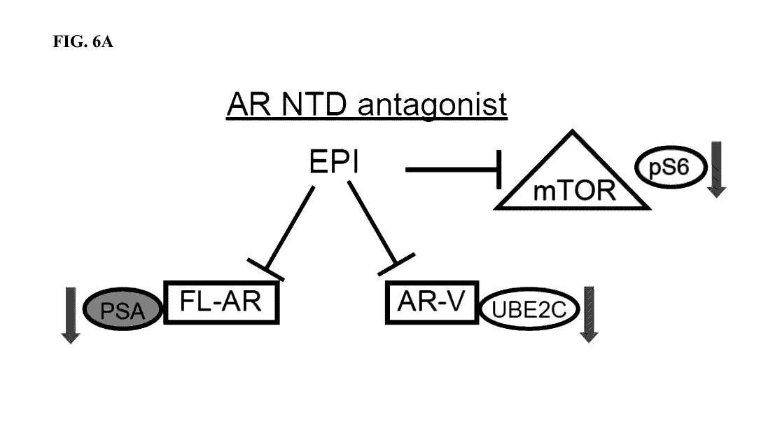

In vivo, Compound A and BEZ-235 monotherapies reduced protein levels of FKBP5, a FL-AR target gene, and UBE2C, an AR-V7 target gene thereby supporting blocking the transcriptional activities of FL-AR and AR-Vs. Immunohistochemical analysis of these same harvested xenografts revealed that Compound A and BEZ-235 reduced levels of UBE2C and pS6 staining (FIG. 5A), which were consistent with western blot data. Also, Compound A significantly decreased proliferation (FIG. 5B) and increased apoptosis (FIG. 5C) as indicated with staining of Ki67 and TUNEL, respectively. In summary, the combination therapy with Compound A and BEZ-235 significantly reduced CRPC tumor growth both in vitro and in vivo.

For FIGS. 5B and 5C, at least 3000 cells per xenograft were counted. Cells that were positive for Ki67 or TUNEL staining were counted in sections from 3 xenografts per treatment. The total number of cells counted was as follows: 4,712 (vehicle, Ki67), 4,833 (Compound A; EPI, Ki67), 5,167 (BEZ-235; BEZ, Ki67), 4123 (combination, Ki67), 4502 (vehicle, TUNEL), 3733 (Compound A, TUNEL), 4109 (BEZ-235, TUNEL) and 3715 (combination, TUNEL). Error bar represent the mean±SEM. One-Way ANOVA post hoc Bonferroni's multiple comparison test, *p<0.05; ***p<0.001; **** p<0.0001.

### Discussions

AR splice variants are a potential mechanism of resistance to abiraterone and enzalutamide in CRPC (Li, Y. et al., Cancer Research 2013, 73, 483-489; Yu, Z. et al. Clin. Cancer Res. 2014, 20, 1590-1600; Liu, L. L. et. al., Oncogene 2014, 33, 3140-3150). AR-NTD targeting drugs have benefits over drugs targeting the AR-LBD because the NTD is essential for the transcriptional activities of both FL-AR and AR-Vs. Antagonists of AR-NTD, such as Compound A, could therefore provide therapeutic responses for CRPC patients with malignancies that express constitutively active AR splice variants and are resistant to abiraterone or antiandrogens. In addition to AR, the PI3K/Akt/mTOR pathway is implicated as a potential driver of CRPC (Bitting, R. L. et. al. Endocrine-Related Cancer 2013, 20, R83-99; Zhang, W. et al. Cancer Research 2009, 69, 7466-7472; Edlind M. P. et al. Asian Journal of Andrology 2014, 16, 378-386). Previous reports have shown therapeutic benefits for the treatment of CRPC by a combination of antiandrogen with an inhibitor of PI3K/Akt/mTOR (Carver B. S. et al Cancer Cell 2011, 19, 575-586; Zhang W. et al Cancer Research 2009, 69, 7466-7472; Thomas, C. et. al., Molecular Cancer Therapeutics 2013, 12, 2342-2355). However, those studies focused on cross-talk with FL-AR and PI3K/Akt/mTOR pathways. Since CRPC that is resistant to antiandrogens and abiraterone has been shown to be correlated to expression of constitutively active AR splice variants, it is of interest to investigate therapeutic effects and mechanisms by combination treatments using an inhibitor of both FL-AR and AR-Vs, such as Compound A, with an inhibitor of PI3K/Akt/mTOR. The examples showed the following:

1) A low, non-toxic concentration of BEZ-235 (15 nM) that did not inhibit pAkt was a potent inhibitor of mTOR;

2) Inhibition of mTOR caused an increase in levels of FL-AR (protein) and its target gene PSA (protein and transcript);

3) Inhibition of mTOR also increased levels of AR-Vs, but decreased endogenous expression of its target genes such as UBE2C, CDC20, and Akt1;

4) Inhibition of mTOR decreased the proliferation of enzalutamide-resistant human prostate cancer cells which is considered to be driven by AR-V7. Combination therapy to block mTOR and the AR-NTD provided significantly better suppression of proliferation than individual monotherapies; and

5) Co-targeting PI3K/Akt/mTOR and AR-NTD in vivo was superior to monotherapies and sufficient to suppress FL-AR and AR-Vs transcriptional activities, and decrease the growth of enzalutamide-resistant CRPC xenografts.

Without bound by any theory, together, these findings support the rationale for co-targeting mTOR and AR-NTD (blocks both FL-AR and AR-Vs) signaling pathways for the treatment of CRPC.

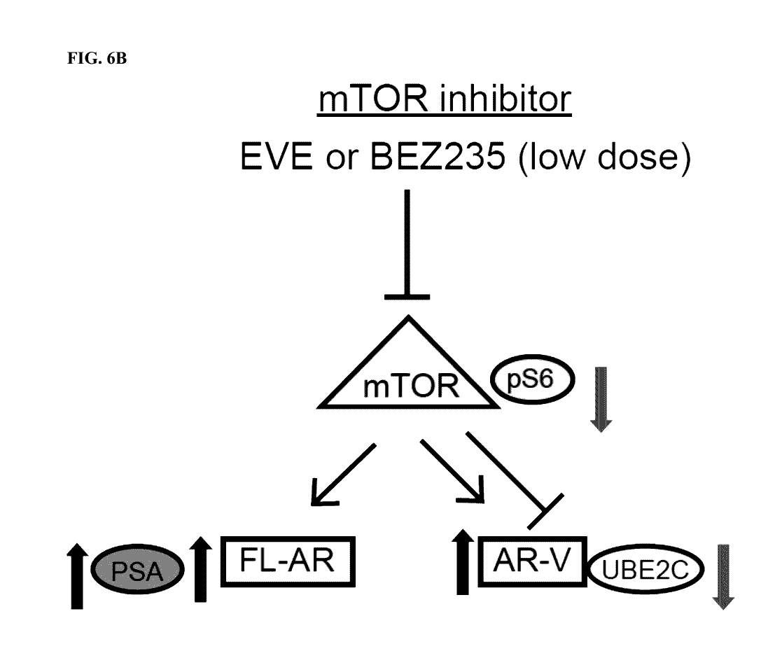

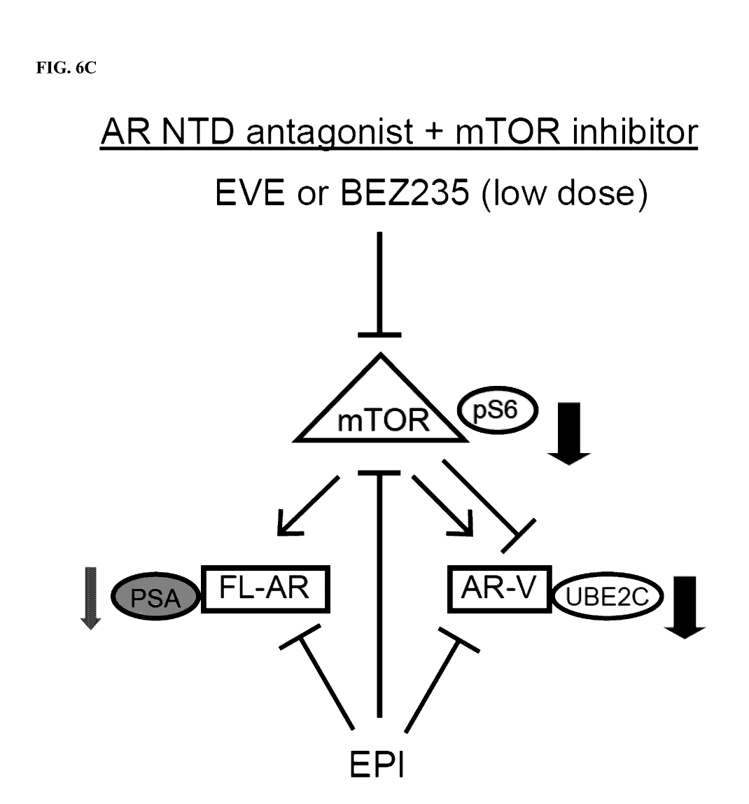

Previous work has implicated that inhibition of FL-AR activates Akt through reducing levels of PHLPP (Carver B. S. et al Cancer Cell 2011, 19, 575-586). Here, enzalutamide and Compound A both decreased mTOR-regulated pS6 while androgen increased pS6. These data suggest that FL-AR regulates mTOR activity, which is consistent with recent studies (Wu, Y. et. al., Anticancer Research 2010, 30, 3895; Munkley, J. et. al., Oncotarget 2014, 5, 131-139). Importantly, here levels of AR were increased by both BEZ-235 and also everolimus without decreasing pAkt. This suggests that mTOR plays an important role in regulating AR protein levels and that Akt was not directly involved. Liu et al reported that PI3K/Akt inhibitors had various effects on AR protein levels in four human prostate cancer cell lines through Akt-independent mechanism (Liu, L. et al PloS One 2014, 9, e108780). It has been reported that both surgical and chemical castration had no effect on the activation of Akt and mTOR (Zhang, W. et al. Cancer Research 2009, 69, 7466-7472). Here BEZ-235 increased protein levels of FL-AR and AR-V7 without concomitant increases in levels of their respective transcripts. Thus, inhibition of mTOR may regulate AR protein levels through post-translational modifications (Cinar, B. et. al., Cancer Research 2005, 65, 2547), such as possibly phosphorylation, acetylation or ubiquitination. Taken together, our results suggest the possibility of an alternative mechanism of cross-talk between these pathways apart from a reciprocal feedback regulation of Akt and FL-AR signaling. A hypothetical model showing cross-talk mechanisms among FL-AR, AR-V and mTOR signaling pathway is shown in FIGS. 6A-6C. In FIGS. 6A-6C, lines represent effects on activity and thick arrows represent changes in levels of expression. Without bound to any theory, in FIG. 6B, levels of AR-V7 target genes such as UBE2C are inhibited possibly by decreased transactivation of AR NTD or other unidentified mechanisms.

Here an important observation was that inhibition of mTOR decreased the expression of ARV7 regulated genes, such as UBE2C, CDC20, and Akt1 while it increased expression of PSA, a gene regulated by FL-AR. This increase in FL-AR transcriptional activity was not by a mechanism of BEZ-235-induced transactivation of the AR as determined using the AR-NTD transactivation assay thereby suggesting that such increased transcriptional activities were more likely due to induced levels of AR protein. Consistent with this interpretation, BEZ-235 had no effect on the transcriptional activities of ectopic AR-V567es or AR-V7 in Cos-1 cells. However, cell-specific responses to inhibition of mTOR would not be unexpected due to variation in signaling pathways in different cell lines.

Up-regulation of the FL-AR pathway was, at least in part, blocked here by enzalutamide and Compound A, which supports a rational that co-targeting both PI3K/Akt/mTOR and FL-AR should achieve better efficacy. However, a clinical trial using a combination of everolimus and bicalutamide to block those pathways failed to achieve a better response when compared to bicalutamide monotherapy (Nakabayashi, M. et. al., BJU International 2012, 110, 1729-1735). Without bound by any theory, a possible explanation of the lack of efficacy could be that the FL-AR signaling pathway was not directly related to the CRPC growth observed and perhaps those tumors were driven by AR splice variants which would not be impacted by an inhibitor of the AR-LBD, such as bicalutamide. Consistent with this notion, enzalutamide did not inhibit the growth of LNCaP95 cells, despite that it effectively blocked FL-AR transcriptional activity. Most importantly, compound A and BEZ-235, but not enzalutamide, reduced levels of UBE2C, an AR-V7 target gene.

In conclusion, our findings demonstrate that co-targeting mTOR and AR-NTD to block both FL-AR and AR-Vs showed maximum antitumor efficacy in PTEN-negative enzalutamide-resistant CRPC with acceptable tolerability. Since AR-LBD targeting drugs may have limited or no effect on AR-Vs, this novel approach may provide a therapeutic advantage for CRPC patients that are resistant to abiraterone or antiandrogens by a mechanism involving expression of AR-V.

Although various embodiments of the invention are disclosed herein, many adaptations and modifications can be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A pharmaceutical combination comprising a therapeutically effective amount of a compound of formula (I), or a pharmaceutically acceptable salt, tautomer or stereoisomer thereof, and at least one additional therapeutically active agent selected from the group consisting of inhibitors of PI3K/AKT/mTOR pathway, agents associated with the treatment of prostate cancer, and anticancer agents; wherein: 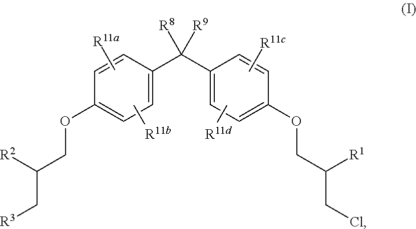
R¹ is hydroxyl or —OC(═O)R¹³;
R² is hydroxyl or —OC(═O)R¹³;
R³ is hydroxyl, halogen, or —OC(═O)R¹³;
R⁸ and R⁹ are each independently H, or C₁-C₃ alkyl;
R^{11a}, R^{11b}, R^{11c} and R^{11d} are each independently H or halogen;
R¹³ is C₁-C₆ alkyl; and
wherein, halo is selected from the group consisting of F, Cl, Br, and I.

2. The pharmaceutical combination of claim 1, wherein the compound is selected from the group consisting of:

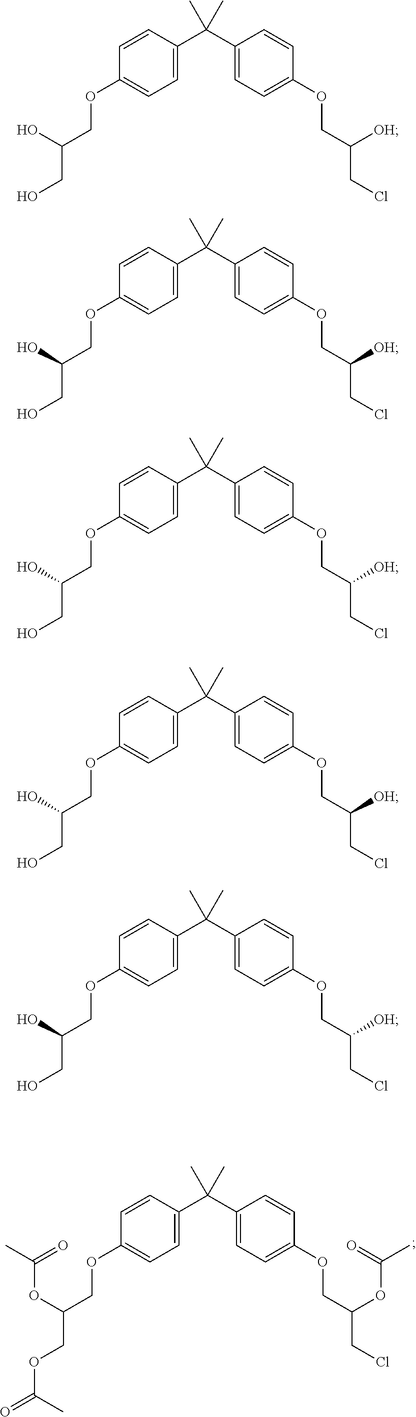 [SMILES: CC(C)(c1ccc(OCC(O)CO)cc1)c1ccc(OCC(O)CCl)cc1 ; CC(C)(c1ccc(OC[C@H](O)CO)cc1)c1ccc(OC[C@@H](O)CCl)cc1 ; CC(C)(c1ccc(OC[C@H](O)CCl)cc1)c1ccc(OC[C@@H](O)CO)cc1 ; CC(C)(c1ccc(OC[C@@H](O)CO)cc1)c1ccc(OC[C@@H](O)CCl)cc1 ; CC(C)(c1ccc(OC[C@H](O)CO)cc1)c1ccc(OC[C@H](O)CCl)cc1 ; CC(=O)OCC(COc1ccc(C(C)(C)c2ccc(OCC(CCl)OC(C)=O)cc2)cc1)OC(C)=O]

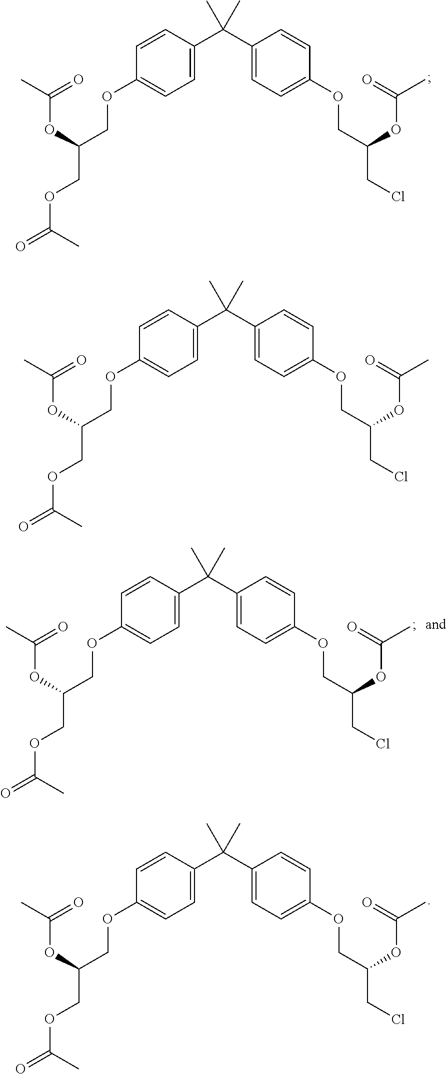

3. The pharmaceutical combination of claim 1, wherein the compound of formula (I) and the at least one additional therapeutically active agent are in single dosage form or in separate dosage forms.

4. The pharmaceutical combination of claim 3, wherein the separate dosage forms are administered via same mode of administration or different modes of administration.

5. The pharmaceutical combination of claim 4, wherein the separate dosage forms are co-administered via simultaneous administration, sequential administration, overlapping administration, interval administration, continuous administration, or a combination thereof.

6. The pharmaceutical combination of claim 1, wherein the at least one additional therapeutically active agent is an inhibitor of PI3K/AKT/mTOR pathway.

7. The pharmaceutical combination of any one of claim 6, wherein the inhibitor of PI3K/AKT/mTOR pathway is a dual PI3K/mTOR inhibitor.

8. The pharmaceutical combination of claim 7, wherein the dual PI3K/mTOR inhibitor is selected from the group consisting of: BEZ-235 (Dactolisib), BEZ-235, XL-765, PF-4691502, GSK-2126458, GDC-0980 and PKI-587.

9. The pharmaceutical combination of claim 8, wherein the dual PI3K/mTOR inhibitor is BEZ-235.

10. The pharmaceutical combination of claim 1, which is a pharmaceutical formulation further comprising a pharmaceutically acceptable excipient or a pharmaceutically acceptable carrier.

11. The pharmaceutical combination of claim 1, further comprising a second additional therapeutically active agent.

12. The pharmaceutical combination of claim 11, wherein the second additional therapeutically active agent is selected from the group consisting of: selected from the group consisting of enzalutamide, galeterone, ARN-509 (4-(7-(6-cyano-5-(tlifluoromethyl)pylidin-3-yl)-8-oxo-6-thioxo-5,7-diazaspiro[3,4]octan-5-yl)-2-tluoro-N-methylbenzamide), abiraterone, bicalutamide, nilutamide, flutamide, cyproterone acetate, docetaxel, bevacizumab, OSU-HDAC42 ((S)-(+)-N-Hydroxy-4-(3-methyl-2phenyl-butyrylamino)benzamide, monoclonal antibody against the vascular integrin ανβ3, sunitumib, ZD-4054 (zibotentan), cabazitaxel (XRP-6258), MDX-010 (ipilimumab), OGX 427 (apatorsen), OGX 011 (custirsen), finasteride, dutasteride, turosteride, bexlosteride, izonsteride, FCE 28260 ((1S,3aS,3bS,5aR,9aR,9bS,11aS)-9a,11a-dimethyl-7-oxo-N-(1,1,1-trifluoro-2-phenylpropan-2-yl)-1,2,3,3a,3b,4,5,5a,6,9b,10,11-dodecahydroindeno[5,4-f]quinoline-1-carboxamide), SKF105,111 (17β-(Di-isopropyl-aminocarbonyl)androsta-3,5-diene-3-carboxylic acid), radium 233, ODM-201, and related compounds thereof.

13. A method for treating a condition or disease that is responsive to modulation of androgen receptor activity, comprising administering to the subject, a therapeutically effective amount of a compound of formula (I); 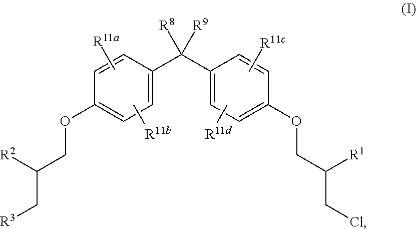
or a pharmaceutically acceptable salt, tautomer or stereoisomer thereof;
wherein
R¹ is hydroxyl or —OC(═O)R¹³;
R² is hydroxyl or —OC(═O)R¹³;
R³ is hydroxyl, halogen, or —OC(═O)R¹³;
R⁸ and R⁹ are each independently H, or C₁-C₃ alkyl;
R^{11a}, R^{11b}, R^{11c} and R^{11d} are each independently H or halogen;
R¹³ is C₁-C₆ alkyl; and
wherein, halo is selected from the group consisting of F, Cl, Br, and I;
and administering of at least one additional therapeutically active agent selected from P inhibitors of PI3K/AKT/mTOR pathway, agents associated with the treatment of prostate cancer, and anticancer agents, before, during, or after the subject has been administered a compound of formula (I).

14. The method of claim 13, wherein the condition or disease is selected from the group consisting of: prostate cancer, breast cancer, ovarian cancer, endometrial cancer, salivary gland carcinoma, hair loss, acne, hirsutism, ovarian cysts, polycystic ovary disease, precocious puberty, spinal and bulbar muscular atrophy, and age-related macular degeneration.

15. The method of claim 14, wherein the condition or disease is prostate cancer.

16. The method of claim 14, wherein the condition or disease is castration resistant prostate cancer.

17. The method of claim 14, wherein the condition or disease is androgen-dependent prostate cancer or androgen-independent prostate cancer.

18. The method of claim 14, wherein the condition or disease is breast cancer.

19. A method for reducing or preventing tumor growth, comprising contacting tumor cells with a therapeutically effective amount of a compound of formula (I); 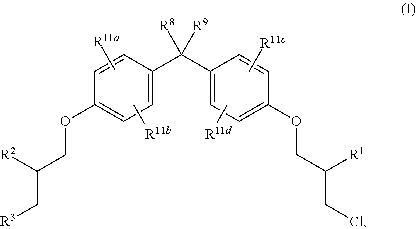
or a pharmaceutically acceptable salt, tautomer or stereoisomer thereof;
wherein
R¹ is hydroxyl or —OC(═O)R¹³;
R² is hydroxyl or —OC(═O)R¹³;
R³ is hydroxyl, halogen, or —OC(═O)R¹³;
R⁸ and R⁹ are each independently H, or C₁-C₃ alkyl;
R^{11a}, R^{11b}, R^{11c} and R^{11d} are each independently H or halogen;
R¹³ is C₁-C₆ alkyl; and
wherein, halo is selected from the group consisting of F, Cl, Br, and I;
and contacting of at least one additional therapeutically active agent selected from inhibitors of PI3K/AKT/mTOR pathway, agents associated with the treatment of prostate cancer, and anticancer agents, before, during, or after the subject has been administered a compound of formula (I).

20. The method of claim 19, wherein the tumor cell is selected from the group consisting of: prostate cancer, breast cancer, ovarian cancer, endometrial cancer, and salivary gland carcinoma.

21. The method of claim 19, wherein the tumor is tumor of the prostate cancer.

22. The method of claim 19, wherein the tumor is tumor of the castration resistant prostate cancer.

23. The method of claim 19, wherein the tumor is androgen-dependent prostate cancer or androgen-independent prostate cancer.

24. The method of claim 19, wherein the tumor is breast cancer.

25. The method of claim 19, wherein the reducing or preventing tumor growth is in vivo or in vitro.

26. The pharmaceutical combination of claim 6, wherein the inhibitor of PI3K/AKT/mTOR pathway is selected from: ridaforolimus, everolimus, temsirolimus, or gedatolisib.

27. The pharmaceutical combination of claim 1, wherein the at least one additional therapeutically active agent is an anticancer agent.

28. The pharmaceutical combination of claim 27, wherein the anticancer agent is sirolimus.
